(19) United States
(12) Patent Application Publication
(10) Pub. No.: US 2008/0221103 A1
(43) Pub. Date: Sep. 11, 2008

(54) New heterocyclic compounds

(75) Inventors: Ganapavarapu V.R. Sharma, Chennai (IN); Gaddam O. Reddy, Chennai (IN); Sriram Rajagopal, Chennai (IN); Uma Ramachandran, Chennai (IN); Sukunath Narayanan, Chennai (IN); Nagalakshmi Pichika, Chennai (IN); Venkatesh Nemmara Viswanathan, Chennai (IN); Lavanya Andiappan, Chennai (IN); Saravanan Thirunavukkarasu, Chennai (IN)

Correspondence Address: OLIFF & BERRIDGE, PLC, P.O. BOX 320850, ALEXANDRIA, VA, 22320-4850, US

(73) Assignee: ORCHID RESEARCH LABORATORIES LTD., CHENNAI (IN)

(21) Appl. No.: 12/073,635

(22) Filed: Mar. 7, 2008

(30) Foreign Application Priority Data
Mar. 9, 2007 (IN) 488/CHE/2007

(51) Int. Cl. A61K 31/5377; C07D 239/24; C07D 413/14; A61K 31/435; A61P 37/06

(52) U.S. Cl. 514/235.8; 544/319; 544/328; 544/122; 514/256

(57) ABSTRACT

Provided herein are heterocyclic compounds of the general formula (I), their derivatives, analogs, tautomeric forms, stereoisomers, polymorphs, hydrates, solvates, pharmaceutically acceptable salts and compositions, metabolites and prodrugs thereof, wherein R¹, R², R³, R⁴ and R⁵ are as described herein. Further described herein in particular are heterocyclic compounds of the formula (I) for treating various diseases. and disorders by administering in a patient one or more TNF-α, Thromboxane synthase, 5-LOX, and PDE4 inhibitors. In particular described herein are methods for treating immunological diseases, inflammation, pain disorder, rheumatoid arthritis; osteoporosis; multiple myeloma; uveititis; acute and chronic myelogenous leukemia; ischemic heart disease; atherosclerosis; cancer; ischemic-induced cell damage; pancreatic beta cell destruction; osteoarthritis; rheumatoid spondylitis; gouty arthritis; inflammatory bowel disease; adult respiratory distress syndrome (ARDS); psoriasis; Crohn's disease; allergic rhinitis; ulcerative colitis; anaphylaxis; contact dermatitis; muscle degeneration; cachexia; asthma; bone resorption diseases; ischemia reperfusion injury; brain trauma; multiple sclerosis; sepsis; septic shock; toxic shock syndrome; fever, and myalgias due to infection and diseases mediated by HIV-1; HIV-2; HIV-3; cytomegalovirus (CMV); influenza; adenovirus; the herpes viruses (including HSV-1, HSV-2) and herpes zoster viruses in a mammal comprising administering an effective amount of a compound of formula (I).

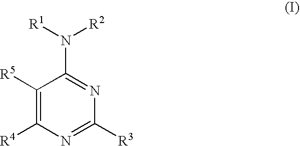

### FIELD

Described are novel heterocyclic compounds of the general formula (I) their derivatives, analogs, tautomeric forms, stereoisomers, polymorphs, hydrates, solvates, pharmaceutically acceptable salts, pharmaceutical compositions, metabolites and prodrugs thereof having significant in vitro TNF-α inhibitory activity useful for the treatment of various inflammatory diseases such as arthritis, inflammatory bowel disease, psoriasis, asthma, COPD, cancer etc., initiated by the excess production of TNF-α.

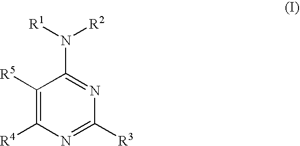

Provided herein are process for the preparation of the above said novel heterocyclic compounds of the general formula (I), their derivatives, analogs, stereoisomers, polymorphs, hydrates, solvates, pharmaceutically acceptable salts, pharmaceutical compositions, metabolites and prodrugs thereof.

The novel heterocyclic compounds provided herein are useful for the treatment of inflammation and immunological diseases. Particularly the compounds described herein are useful for the treatment of cancer, inflammation and immunological diseases those mediated by cytokines such as TNF-α, IL-1, IL-6, IL-1β, IL-8, IL-12, cyclooxygenases such as COX-1, COX-2 and COX-3, lipoxygenases such as 5-LOX, 12-LOX, and 15-LOX, and thromboxane. The compounds described herein are useful as PDE4 inhibitors, and are useful for treating PDE4 mediated diseases such as asthma, COPD, IBD, arthritis, psoriasis and the like. More particularly, the compounds described herein are useful as dual inhibitors of 5-LOX and thromboxane synthase, and are useful for treating lipoxygenase and thromboxane mediated diseases such as asthma, COPD, IBD, arthritis, psoriasis, cancer and the like. The compounds of the present invention are also useful for the treatment of rheumatoid arthritis; osteoporosis; multiple myeloma; uveititis; acute and chronic myelogenous leukemia; ischemic heart disease, atherosclerosis, cancer, ischemic-induced cell damage, pancreatic β cell destruction; osteoarthritis; rheumatoid spondylitis; gouty arthritis; inflammatory bowel disease; adult respiratory distress syndrome (ARDS); psoriasis; Crohn's disease; allergic rhinitis; ulcerative colitis; anaphylaxis; contact dermatitis; asthma; muscle degeneration; cachexia; type I and type II diabetes; bone resorption diseases; ischemia reperfusion injury; brain trauma; multiple sclerosis; cerebral malaria; sepsis; septic shock; toxic shock syndrome; fever and myalgias due to infection; and diseases mediated by HIV-1; HIV-2; HIV-3; cytomegalovirus (CMV); influenza; adenovirus; the herpes viruses (including HSV-1, HSV-2) and herpes zoster viruses.

### BACKGROUND

Autoimmune diseases affect millions of people across the world with a number of pathologies associated with autoimmune processes representing a wide and fast-moving area of research and clinical interest (Nature 1985, 318, 665-667; Reumatismo, 2006, 58(2), 94-103; Nature Immunology, 2001, 2, 771-773). Extensive preclinical research has been focused on the regulation of cytokine expression with a particular interest in inflammatory diseases like rheumatoid arthritis (RA); this multidisciplinary effort has contributed to elucidate the roles of cytokines in this and other disabling autoimmune diseases. Tumor necrosis factor alpha (TNF-α) emerged from these studies as a pivotal regulator of expression of other pro-inflammatory cytokines such as Interleukin-1 (IL-1) and Interleukin-6 (IL-6) (Nature Immunol 2001, 2, 759-761; Science 2000, 288, 2351-2354; Proc. Natl. Acad. Sci., USA, 1982, 89, 9784-9788), thus becoming a key target for therapeutic intervention in a redundant cytokine environment.

As already mentioned, blocking excess TNF-αcan be therapeutically useful through its cascade effects on other pro-inflammatory cytokines. Towards this end several approaches have resulted in the development of biological molecules as anti-TNF therapy. Etanercept (Enbrel), an engineered soluble receptor of TNF-α with clinical application was originally approved by the US FDA in 1998 to treat the painful joint swelling and deterioration caused by rheumatoid arthritis (Reumatismo 2006, 58(2), 94-103). There are several other biologicals developed since then like Adalimumab (Humira) and Infliximab (Remicade). However, the limitation in the development of these molecules is the cost for the production of these molecules and the side effects caused by their use. Alternative approaches necessitated the invention of small molecule inhibitors.

IL-6 is a protein belonging to the group of cytokines, which proved to play a key role in the organism's immune response and haematopoiesis stimulation. Many biological functions have, in fact, been found for IL-6 in the hematopoietic and lymphoid system, in the liver and in other target organs and cells. Some of these functions are beneficial, while others are related to pathological states. Among the latter functions, IL-6 has been found to be a growth factor for multiple myeloma cells; anti-IL-6 antibodies were shown to transiently block myeloma cell proliferation in a leukemic patient. Elevated levels of IL-6 have been correlated with autoimmune and inflammatory diseases (U.S. Pat. No. 5,527,546/1996; U.S. Pat. No. 6,004,813/1999) such as rheumatoid arthritis, glomerulonephritis, psoriasis, and Castelman's disease. IL-6 has also been shown to play a direct role in bone loss and hypercalcemia. The development of inhibitors of IL-6 activity has therefore been the subject of active research.

IL-1 is one of the first cytokines ever described. Its initial discovery was as a factor that could induce fever, control lymphocytes, increases the number of bone marrow cells and cause degeneration of bone joints. At this time, IL-1 was known under several other names including endogenous pyrogen, lymphocyte activating factor, haemopoetin-1 and mononuclear cell factor, amongst others. It was later confirmed that IL-1 was actually composed of two distinct proteins, now called IL-1α and IL-1β (Developmental and Comparative Immunology, 2004, 28, (5), 395-413). These belong to a family of cytokines known as the interleukin-1 superfamily. Both IL-1α and IL-1β are produced by macrophages, monocytes and dendritic cells. They form an important part of the inflammatory response of the body against infection. These cytokines increase the expression of adhesion factors on endothelial cells to enable transmigration of leukocytes, the cells that fight pathogens, to sites of infection and re-set the hypothalamus thermoregulatory center, leading to an increased body temperature which expresses itself as fever, IL-1 is therefore called an endogenous pyrogen. The increased body temperature helps the body's immune system to fight infection. IL-1 is also important in the regulation of hematopoiesis. IL-1 inhibitors are being developed for the treatment of autoimmune diseases like rheumatoid arthritis, wherein IL-1 plays a key role. One such inhibitor that is commercially produced is Anakinra, a human recombinant form of IL-1RA (IL-1 receptor antagonist).

Elevated levels of TNF-α and/or IL-1 over basal levels have been implicated in mediating or exacerbating a number of disease states including rheumatoid arthritis; osteoporosis; multiple myeloma; uveititis; acute and chronic myelogenous leukemia; pancreatic β cell destruction; osteoarthritis; rheumatoid spondylitis; gouty arthritis; inflammatory bowel disease; adult respiratory distress syndrome (ARDS); psoriasis; Crohn's disease; allergic rhinitis; ulcerative colitis; anaphylaxis; contact dermatitis; asthma; muscle degeneration; cachexia; type I and type II diabetes; bone resorption diseases; ischemia reperfusion injury; atherosclerosis; brain trauma; multiple sclerosis; cerebral malaria; sepsis; septic shock; toxic shock syndrome; fever, and myalgias due to infection. HIV-1, HIV-2, HIV-3, cytomegalovirus (CMV), influenza, adenovirus, the herpes viruses (including HSV-1, HSV-2), and herpes zoster are also exacerbated by TNF-α.

IL-8 has been implicated in exacerbating and/or causing many disease states in which massive neutrophil infiltration into sites of inflammation or injury (e.g., ischemia) is mediated; chemotactic nature of IL-8, including, but is not limited to, the following: asthma, inflammatory bowel disease, psoriasis, adult respiratory distress syndrome, cardiac and renal reperfusion injury, thrombosis and glomerulonephritis. In addition to the chemotaxis effect on neutrophils, IL-8 also has ability to activate neutrophils. Thus, reduction in IL-8 levels may lead to diminish neutrophil infiltration. IL-12 is linked with autoimmunity. Administration of IL-12 to people suffering from autoimmune diseases was shown to worsen the autoimmune phenomena. This is believed to be due to its key role in induction of Th1 immune responses. In contrast, IL-12 gene knockout in mice or a treatment of mice with IL-12 specific antibodies ameliorated the disease.

COX converts arachidonic acid to prostaglandin H2, the precursor of the series-2 prostanoids. Currently three COX isoenzymes are known viz., COX-1, COX-2 and COX-3. COX-3 is a splice variant of COX-1 which retains intron one and has a frameshift mutation, thus some prefer the name COX-1b or COX-1 variant (COX-1v), (N. V. Chandrasekharan et. al., Proc. Natl, Acad, Sciences USA, 2002, 99(21), 13926-139231; T D Warner et al., Proc. Natl, Acad, Sciences USA, 2002, 99(21), 13371-13373; R J Soberman et al., J. Clin. Invest., 2003, 111, 1107-1113)

Different tissues express varying levels of COX-1 and COX-2. Although both enzymes act basically in the same fashion, selective inhibition can make a difference in terms of side effects. COX-1 is considered a constitutive enzyme, being found in most mammalian cells. More recently it has been shown to be up regulated in various carcinomas and to have a central role in tumorigenesis. COX-2 on the other hand is undetectable in most normal tissues. It is an inducible enzyme becoming abundant in activated macrophages and other cells at sites of inflammation. (N. V. Chandrasekharan et. al., Proc. Natl, Acad, Sciences USA, 2002, 99(21), 13926-139231,

The lipid mediator thromboxane A2 (Tx) is a biologically active metabolite of arachidoic acid, which is synthesized from prostaglandin endoperoxide via thromboxane A synthase. Once formed, Tx which has a very short half life (t_{l/2}=30 s), is rapidly broken down by hydrolysis to the inactive thromboxane B2.

Tx is produced locally by platelets, macrophages, vascular smooth muscle cells of arteries and veins, endothelial cells and human cardiac atrial tissue. In addition to its major role as a powerful platelet aggregator, Tx is a potent vasoconstrictor, stimulator of vascular smooth muscle cell growth and is a positive inotropic mediator in the heart. Interestingly, increased production (approximately 10 ng ml71, compared with 1±2 pg ml71 in normal healthy plasma) of Tx has been implicated in cardiac pathology, including ischaemic heart disease, pulmonary hypertension and heartfailure (British Journal of Pharmacology, 2001, 134, 1385-1392). In addition, thromboxane was also implicated in diseases such as asthma, COPD, and IBD.

The lipoxygenases are non-heme, non-sulfur iron dioxygenases that act on lipid substrates containing one or more 1,4-pentadiene moieties to form hydroperoxides. 5-Lipoxygenase is a key enzyme that catalyses the first two steps in the oxygenation of arachidonic acid, which is converted to biologically active leukotrienes, namely leukotriene B4 and cysteinyl leukotrienes. Leukotrienes play important role in the pathophysiology of inflammatory/allergic diseases including bronchial asthma, allergic rhinitis, urticaria, atopic dermatitis, chronic obstructive pulmonary disease. Incidences of allergic/inflammatory diseases are on the rise world over (US20080021080).

Phosphodiesterases (“PDE”) are a family of enzymes that metabolise 3′ 5′ cyclic nucleotides to 5′ nucleoside monophosphates thereby terminating camp second messenger activity. A particular phosphodiesterase, phosphodiesterase-4 (“PDE4” also known as “PDE IV”), which is a high affinity, camp specific, type IV PDE, has generated interest as potential target for the development of novel anti-asthmatic and anti-inflammatory compounds. PDE4 is known to exist as at least four isoenzymes, each of which is encoded by a distinct gene. Each of the four known PDE4 gene products is believed to play varying roles in allergic and/or inflammatory responses. Thus it is believed that inhibition of PDE4, particularly the specific PDE4 isoforms that produce detrimental responses, can beneficially affect allergy and inflammation symptoms. It would be desirable to provide a method of treatment of rheumatoid arthritis by administering compounds and compositions that inhibit PDE4 activity.

A major concern with the use of PDE4 inhibitors is the side effect of emesis which has been observed for several candidate compounds as described in the patents U.S. Pat. No. 5,622,977, WO 99/50262, U.S. Pat. No. 6,410,563, and U.S. Pat. No. 5,712,298. It was also described the wide variation of the severity of the undesirable side effects exhibited by various compounds. There is a great interest and research of therapeutic PDE4 inhibitors as described in the above mentioned patents and references cited therein.

I. US 2005245508 A1 discloses substituted pyrimidines as represented by the following general structure for treating malignant gliomas, by administering inhibitors of TGF-β, the TGF-β signaling pathway, including molecules preferably ITGF-β receptor (TGFβ-R1).

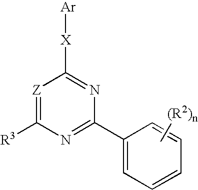

Wherein, Ar represents an optionally substituted aromatic or optionally substituted heteroaromatic moiety containing 5-12 ring members wherein, heteroaromatic moiety contains one or more N, O or S. X is NR¹ wherein, R¹ is H, alkyl (C₁-C₈), alkenyl (C₂-C₈) or alkynyl (C₂-C₈); R² is independently alkyl, alkenyl, alkynyl, acyl, aryl, alkylaryl, aroyl, or hetero forms thereof and each may be unsubstituted or substituted by 1-3 substituents selected independently from groups such as halo, NR₂, OR, SR, cyano, trifluoromethyl, NO₂ and the like. Z is CR⁴. R³ and R⁴ is independently hydrogen, alkyl, alkenyl, alkynyl, acyl, aryl, alkylaryl, aroyl, O-aryl, O-aroyl or heteroforms there of and each may be unsubstituted or substituted by 1-3 substituents selected independently from groups such as halo, OR, NR₂, SR, CN, CF₃ or NO₂.

II. WO 2005047268 A2 discloses substituted pyrimidines as represented by the following general structure.

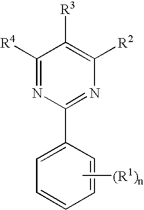

Wherein n is 0 to 5. R¹ is selected from alkyl, nitro, halo, cyano, mercapto, hydroxy, formyl, optionally substituted alkyl, alkenyl, alkynyl and the like. R² is selected from group consisting of alkyl, alkenyl, aryl, heteroaryl, cycloalkyl and the like. R³ is selected from the group consisting of halo, cyano, nitro, hydroxy, formyl mercapto and the like. R² and R³ together with the carbon atom to which they are attached to form an optionally substituted cycloalkyl ring, heterocyclyl ring and an optionally substituted cycloalkenyl ring. R⁴ is selected from group consisting of hydrogen, halo, cyano, nitro, hydroxy, formyl and mercapto, optionally substituted alkyl and the like.

III. Also preparation of similar compounds as mentioned above with minor modifications was reported in the patents WO 2006076442, WO 2006100095 A1, US 2005049247, US 2004009981 and US 2003225073.

### OBJECTIVE

One objective herein is to provide novel heterocyclic compounds of the general formula (I) their derivatives, analogs, tautomeric forms, stereoisomers, polymorphs, hydrates, solvates, pharmaceutically acceptable salts and compositions, metabolites and prodrugs thereof. Also included is a method of treatment of immunological diseases, inflammation, pain disorder, rheumatoid arthritis; osteoporosis; multiple myeloma; uveititis; acute and chronic myelogenous leukemia; ischemic heart disease; atherosclerosis; cancer; ischemic-induced cell damage; pancreatic beta cell destruction; osteoarthritis; rheumatoid spondylitis; gouty arthritis; inflammatory bowel disease; adult respiratory distress syndrome (ARDS); psoriasis; Crohn's disease; allergic rhinitis; ulcerative colitis; anaphylaxis; contact dermatitis; muscle degeneration; cachexia; asthma; bone resorption diseases; ischemia reperfusion injury; brain trauma; multiple sclerosis; sepsis; septic shock; toxic shock syndrome; fever, and myalgias due to infection and diseases mediated by HIV-1; HIV-2; HIV-3; cytomegalovirus (CMV); influenza; adenovirus; the herpes viruses (including HSV-1, HSV-2) and herpes zoster viruses in a mammal comprising administering an effective amount of a compound of formula (I, Ia) as described above.

Another objective herein is to provide a process for the preparation of the novel heterocyclic compounds of general formula (I, Ia), their derivatives, analogs, tautomeric forms, stereoisomers, polymorphs, hydrates, solvates, pharmaceutically acceptable salts, pharmaceutical compositions, metabolites and prodrugs thereof mentioned above.

### SUMMARY

The present invention relates to novel heterocyclic compounds of the general formula (I),

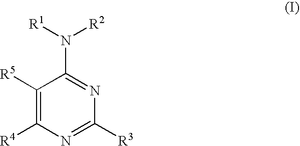

their derivatives, analogs, tautomeric forms, stereoisomers, polymorphs, hydrates, solvates, pharmaceutically acceptable salts, pharmaceutical compositions, metabolites. and prodrugs thereof, wherein R¹ and R² independently represent hydrogen, amino, optionally substituted groups selected from linear or branched alkyl, cycloalkyl alkylsulfonyl; aryl, heteroaryl, nitrogen containing saturated or unsaturated heterocyclyl ring or R¹ and R² can together form an optionally substituted saturated or unsaturated cyclic ring. R³ represents optionally substituted groups selected from linear or branched alkyl, alkyl thio, amino, aryl and heteroaryl. R⁴ represents optionally substituted groups selected from linear or branched alkyl, alkylthio, alkylsulfonyl, alkylsulfinyl, aryl and heteroaryl. R⁵ represents hydrogen, hydroxyl, halogen, nitro, heterocyclyl such as tetrazolyl cyano, carboxylic acid, esters, optionally substituted groups selected from linear or branched alkyl, amino and amide.

### DETAILED DESCRIPTION

The present invention relates to novel heterocyclic compounds of the general formula (I),

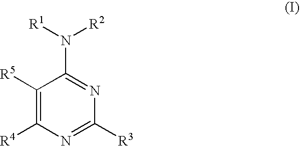

their derivatives, analogs, tautomeric forms, stereoisomers, polymorphs, hydrates, solvates, pharmaceutically acceptable salts, pharmaceutical compositions, metabolites and prodrugs thereof, wherein R¹ and R² independently represent hydrogen, amino group, optionally substituted groups selected from linear or branched alkyl, cycloalkyl, alkylsulfonyl, aryl, heteroaryl; nitrogen containing saturated or unsaturated heterocyclyl rings such as pyrrolinyl, pyrrolidinyl, pyridyl, pyrrolyl, oxazolyl, thiazolyl, imidazolyl, pyrazolyl, oxadiazolyl, thiadiazolyl, tetrazolyl, pyrimidinyl, pyradazinyl, benzimidazolyl, benzoxazolyl, benzothiazolyl, benzopyrrolyl, benzoxadiazolyl, benzothiadiazolyl, quinolinyl, isoquinolinyl, indolyl, dihydropyradazinyl, morpholinyl, thiomorpholinyl, piperazinyl and piperidinyl; or R¹ and R² can together with the nitrogen atom to which they are attached form an optionally substituted saturated or unsaturated cyclic ring such as pyridyl, pyrrolyl, oxazolyl, thiazolyl, imidazolyl, pyrazolyl, oxadiazolyl, thiadiazolyl, tetrazolyl, pyrimidinyl, pyradazinyl, benzimidazolyl, benzoxazolyl, benzothiazolyl, benzopyrrolyl, benzoxadiazolyl, benzothiadiazolyl, quinolinyl, isoquinolinyl, indolyl, dihydropyradazinyl, morpholinyl, thiomorpholinyl, piperazinyl and piperidinyl. R³ represents optionally substituted groups selected from, linear or branched alkyl, alkylthio, amino, aryl and heteroaryl. R⁴ represents optionally substituted groups selected from linear or branched alkyl, alkylthio, alkylsulfonyl, alkylsulfinyl, aryl and heteroaryl. R⁵ represents hydrogen, hydroxyl, halogen, nitro, amino, cyano, amide, carboxylic acid and its derivatives, optionally substituted groups selected from linear or branched alkyl.

Yet another embodiment of the present invention there is provided a novel pyrimidine derivatives of the formula (Ia)

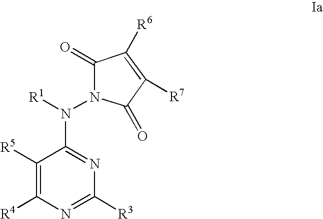

wherein R¹ represent hydrogen; optionally substituted groups selected from linear or branched alkyl such as methyl, ethyl, n-propyl, isopropyl, n-butyl, isobutyl, t-butyl, n-pentyl, isopentyl, hexyl and the like; cycloalkyl such as cyclopropyl, cyclobutyl and the like; amino; alkylsulfonyl such as methylsulfonyl, ethylsulfonyl and the like. R³ represents halogen such as fluorine, chlorine, bromine, iodine and the like; substituted or unsubstituted alkyl, haloalkyl group such as chloromethane, chloroethane, trifluoromethane, trifluoroethane, dichloromethane, dichloroethane and the like; optionally substituted groups selected from linear or branched alkyl; alkoxy group such as methoxy, ethoxy and the like; alkylthio group such as methylthio, ethylthio and the like; alkylsulfinyl group such as methylsulfinyl, ethylsulfinyl and the like; aryl such as phenyl, naphthyl and the like; heterocyclyl such as pyrrolidinyl, thiazolidinyl, oxazolidinyl, morpholinyl, thiomorpholinyl, piperidinyl, piperazinyl, and the like; and heteroaryl such as pyridyl, thienyl, furyl, pyrrolyl, oxazolyl, thiazolyl, imidazolyl, pyrazolyl, oxadiazolyl, thiadiazolyl, tetrazolyl, pyrimidinyl benzopyranyl, benzimidazolyl, benzoxazolyl, benzothiazolyl, benzopyrrolyl, benzoxadiazolyl, benzothiadiazolyl, quinolinyl, isoquinolinyl, benzothienyl, benzofuranyl, indolyl and the like. R⁴ represents optionally substituted groups selected from linear or branched alkyl; alkylthio; alkylsulfonyl; alkylsulfinyl such as methylsulfinyl, ethylsulfinyl and the like; aryl and heteroaryl. R⁵ represents hydrogen; hydroxyl; halogen; nitro; cyano; amide; heterocyclyl groups such as substituted or unsubstituted tetrazolyl carboxylic acid and its derivatives; optionally substituted groups selected from linear or branched alkyl and amino. R⁶ and R⁷ represents hydrogen; halogen; nitro; haloalkyl; optionally substituted groups selected from linear or branched alkyl; amino; aryl; heteroaryl or R⁶ and R⁷ can together form a optionally substituted saturated or unsaturated cyclic ring such as cycloalkyl; aryl; heteroaryl.

When the groups R¹, R², R³, R⁴, R⁵, R⁶ and R⁷ have one or more substitutents, the substituents are selected from halogen; haloalkyl; oxo; nitro; hydroxyl; carboxylic acid; ester; amide; alkyl; alkoxy; amino; aminosulfonyl; heterocyclylalkyl; heterocyclylsulfonyl; alkylthio; mercapto; aryl; heteroaryl and heteroarylalkyl groups; which in turn are optionally substituted by halogen; alkyl; alkoxy; aryl and heteroaryl.

The term analog includes a compound, which differs from the parent structure by one or more C, N, O or S atoms. Hence, a compound in which one of the N atoms in the parent structure is replaced by an S atom is an analog of the former.

The term stereoisomer includes isomers that differ from one another in the way the atoms are arranged in space, but whose chemical formulas and structures are otherwise identical. Stereoisomers include enantiomers and diastereoisomers.

The term tautomers include readily interconvertible isomeric forms of a compound in equilibrium. The enol-keto tautomerism is an example.

The term polymorphs include crystallographically distinct forms of compounds with chemically identical structures.

The term pharmaceutically acceptable solvates includes combinations of solvent molecules with molecules or ions of the solute compound.

The term derivative refers to a compound obtained from a compound according to formula (I, Ia), an analog, tautomeric form, stereoisomer, polymorph, hydrate, pharmaceutically acceptable salt or pharmaceutically acceptable solvate thereof, by a simple chemical process converting one or more functional groups, such as, by oxidation, hydrogenation, alkylation, esterification, halogenation and the like.

Pharmaceutically acceptable salts of the present invention include alkali metals like Li, Na, and K, alkaline earth metals like Ca and Mg, salts of organic bases such as diethanolamine, α-phenylethylamine, benzylamine, piperidine, morpholine, pyridine, hydroxyethylpyrrolidine, choline hydroxyethylpiperidine, and the like, ammonium or substituted ammonium salts, aluminum salts. Salts also include amino acid salts such as glycine, alanine, cystine, cysteine, lysine, arginine, phenylalanine, guanidine etc. Salts may include acid addition salts where appropriate, which are, sulphates, nitrates, phosphates, perchlorates, borates, hydrohalides, acetates, tartrates, maleates, citrates, succinates, palmoates, methanesulphonates, tosylates, benzoates, salicylates, hydroxynaphthoates, benzenesulfonates, ascorbates, glycerophosphates, ketoglutarates and the like. Pharmaceutically acceptable solvates may be hydrates or comprising other solvents of crystallization such as alcohols.

A term once described, the same meaning applies for it, throughout the patent

The following representative compounds have been prepared following the procedures mentioned above. However, it does not limit the scope of the invention.

Representative compounds include:
1. 4-(3-Methyl-2,5-dioxo-2,5-dihydro-1H-pyrrol-1-ylamino)-6-(methylthio)-2-phenylpyrimidine-5-carbonitrile;
2. 4-(Thiomethyl)-2-(4-methoxyphenyl)-6-(3-methyl-2,5-dioxo-2,5-dihydro-1H-pyrrol-1-ylamino)pyrimidine-5-carbonitrile;
3. 4-Hydrazinyl-2-(4-methoxyphenyl)-6-(methylthio)pyrimidine-5-carbonitrile;
4. 5-(5-Cyano-4-hydrazinyl-6-(methylthio)pyrimidin-2-yl)-2-methoxybenzenesulfonamide;
5. 4-(3-Methyl-2,5-dioxo-2,5-dihydro-1H-pyrrol-1-ylamino)-6-(methylthio)-2-(2,4,6-trimethoxyphenyl)pyrimidine-5-carbonitrile;
6. 5-(5-Cyano-4-(3-methyl-2,5-dioxo-2,5-dihydro-1H-pyrrol-1-ylamino)-6-(methylthio)pyrimidin-2-yl)-2-methoxybenzenesulfonamide;
7. 4-Hydrazinyl-6-(methylthio)-2-(4-(trifluoromethyl)phenyl)pyrimidine-5-carbonitrile;
8. 3-(5-Cyano-4-hydrazinyl-6-(methylthio)pyrimidin-2-yl)benzenesulfonamide;
9. 4-(3-Methyl-2,5-dioxo-2,5-dihydro-1H-pyrrol-1-ylamino)-6-(methylthio)-2-(4-trifluoromethyl)phenyl)pyrimidine-5-carbonitrile;
10. 3-(5-Cyano-4-(3-methyl-2,5-dioxo-2,5-dihydro-1H-pyrrol-1-ylamino)-6-(methylthio)pyrimidin-2-yl)benzenesulfonamide;
11. 2-(4-Chlorophenyl)-4-(3-methyl-2,5-dioxo-2,5-dihydro-1H-pyrrol-1-ylamino)-6-(methylthio)pyrimidine-5-carbonitrile;
12. 2-(4-(Dimethylamino)phenyl)-4-(3-methyl-2,5-dioxo-2,5-dihydro-1H-pyrrol-1-ylamino)-6-(methylthio)pyrimidine-5-carbonitrile;
13. 4-Hydrazinyl-6-(methylthio)-2-(thiophen-2-yl)pyrimidine-5-carbonitrile;
14. 4-(3-Methyl-2,5-dioxo-2,5-dihydro-1H-pyrrol-1-ylamino)-6-(methylamino)-2-(thiophen-2-yl)pyrimdine-5-carbonitrile;
15. 4-(Thiomethyl)-2-(3-ethoxycarbonyl-phenyl)-6-(3-methyl-2,5-dioxo-2,5-dihydro-1H-pyrrol-1-ylamino)pyrimidine-5-carbonitrile;

16. Ethyl-3-(5-cyano-4-(5-methyl-3,6-dioxo-2,3-dihydropyridazin-1(6H)-yl)-6-(methylthio)pyrimidin-2-yl)benzoate;
17. 4-(3-Methyl-2,5-dioxo-2,5-dihydro-1H-pyrrol-1 ylamino)-6-(methylthio)-2-p-tolylpyrimidine-5-carbonitrile;
18. 2-(2-Chloropyridin-3-yl)-4-(3-methyl-2,5-dioxo-2,5-dihydro-1H-pyrrol-1-ylamino)-6-(methylthio)pyrimidine-5-carbonitrile;
19. 4-(3-Methyl-2,5-dioxo-2,5-dihydro-1H-pyrrol-1-ylamino)-6-(methylthio)-2-(4-(methylthio)phenyl)pyrimidine-5-carbonitrile;
20. 2-(4-((2-Methyl-1H-imidazol-1-yl)methyl)phenyl)-4-(3-methyl-2,5-dioxo-2,5-dihydro-1H-pyrrol-1-ylamino)-6-(methylthio)pyrimidine-5-carbonitrile;
21. 2-(2-Chlorophenyl)-4-(3-methyl-2,5-dioxo-2,5-dihydro-1H-pyrrol-1-ylamino)-6-(methylthio)pyrimidine-5-carbonitrile;
22. 2-(3,4-Dihydroxyphenyl)-4-(3-methyl-2,5-dioxo-2,5-dihydro-1H-pyrrol-1-ylamino)-6-(methylthio)pyrimidine-5-carbonitrile;
23. 2-(2,4-Dichlorophenyl)-4-(3-methyl-2,5-dioxo-2,5-dihydro-1H-pyrrol-1-ylamino)-6-(methylthio)pyrimidine-5-carbonitrile;
24. 2-(2-Fluorophenyl)-4-(3-methyl-2,5-dioxo-2,5-dihydro-1H-pyrrol-1-ylamino)-6-(methylthio)pyrimidine-5-carbonitrile;
25. 2-(3,4-Difluorophenyl)-4-(3-methyl-2,5-dioxo-2,5-dihydro-1H-pyrrol-1-ylamino)-6-(methylthio)pyrimidine-5-carbonitrile;
26. 2-(4-Hydroxyphenyl)-4-(3-methyl-2,5-dioxo-2,5-dihydro-1H-pyrrol-1-ylamino)-6-(methylthio)pyrimidine-5-carbonitrile;
27. 4-(3,4-Dichloro-2,5-dioxo-2,5-dihydro-1H-pyrrol-1-ylamino)-2-(4-methoxyphenyl)-6-(methylthio)pyrimidine-5-carbonitrile;
28. 4-(3,4-Dimethyl-2,5-dioxo-2,5-dihydro-1H-pyrrol-1-ylamino)-2-(4-methoxyphenyl)-6-(methylthio)pyrimidine-5-carbonitrile;
29. 2-(2-Methoxyphenyl)-4-(3-methyl-2,5-dioxo-2,5-dihydro-1H-pyrrol-1-ylamino)-6-(methylthio)pyrimidine-5-carbonitrile;
30. 4-(3-Methyl-2,5-dioxo-2,5-dihydro-1H-pyrrol-1-ylamino)-6-(methylthio)-2-(naphthalen-1-yl)pyrimidine-5-carbonitrile;
31. 2-Methyl-4-(3-methyl-2,5-dioxo-2,5-dihydro-1H-pyrrol-1-ylamino)-6-(methylthio)-2-(naphthalen-1-yl)pyrimidine-5-carbonitrile;
32. 4-(3,4-Dimethyl-2,5-dioxo-2,5-dihydro-1H-pyrrol-1-ylamino)-6-(methylthio)-2-(phenyl)pyrimidine-5-carbonitrile;
33. 4-(3,4-Dichloro-2,5-dioxo-2,5-dihydro-1H-pyrrol-1-ylamino)-6-(methylthio)-2-(phenyl)pyrimidine-5-carbonitrile;
34. 4-(1,3-Dioxoisoindolin-2-ylamino)-6-(methylthio)-2-ylamino)-6-(methylthio)-2-phenylpyrimidine-5-carbonitrile;
35. 4-(3-Methyl-2,5-dioxo-2,5-dihydro-1H-pyrrol-1-ylamino)-6-(methylthio)-2-m-tolylpyrimidine-5-carbonitrile;
36. 4-(Thiomethyl)-2-(thiomethyl)-6-(3-methyl-2,5-dioxo-2,5-dihydro-1H-pyrrol-1-ylamino)pyrimidine-5-carbonitrile;
37. 1-(6-(3-(Aminothioperoxy)-4-methylphenyl)-2-methylpyrimidin-4-ylamino)-3-methyl-1H-pyrrole-2,5-dione;
38. 3-Methyl-1-(2-methyl-6-phenylpyrimidin-4-ylamino)-1H-pyrrole-2,5-dione;
39. 2-(4-Methoxyphenyl)-4-(methylthio)-6-(4-(pyridin-2-yl)piperazin-1-yl)pyrimidine-5-carbonitrile;
40. 4-(1H-imidazol-1-yl)-2-(4-methoxyphenyl)-6-(methylthio)pyrimidine-5-carbonitrile;
41. 2-(4-Methoxyphenyl)-4-(methylthio)-6-(piperazin-1-yl)pyrimidine-5-carbonitrile;
42. 4-(Methylthio)-6-morpholino-2-phenylpyrimidine-5-carbonitrile;
43. 2-(4-Methoxyphenyl)-4-(methylthio)-6-morpholinopyrimidine-5-carbonitrile;
44. 4-(1H-imidazol-1-yl)-6-(methylthio)-2-phenylpyrimidine-5-carbonitrile;
45. 2-(4-Methoxyphenyl)-4-(methylthio)-6-(4-nitro-1H-imidazol-1-yl)pyrimidine-5-carbonitrile;
46. 5-(5-Cyano-4-(methylthio)-6-morpholinopyrimidin-2-yl)-2-methoxybenzenesulfonamide;
47. 2-(4-Methoxy-3-(morpholinsulfonyl)phenyl)-4-(methylthio)-6-morpholinopyrimidine-5-carbonitrile;
48. 5-(5-Cyano-4-(methylthio)-6-(4-(pyridine-2-yl)piperazin-1-yl)pyrimidin-2-yl)-2-methoxybenzenesulfonamide;
49. 5-(5-Cyano-4-(cyclopropylamino)-6-(methylthio)pyrimidin-2-yl)-benzenesulfonamide;
50. 5-(5-Cyano-4-(cyclopropylamino)-6-(methylthio)pyrimidin-2-yl)-2-methoxybenzenesulfonamide;
51. 4-(Methylthio)-2-phenyl-6-(piperazin-1-yl)pyrimidine-5-carbonitrile;
52. 4-(Methylthio)-2-phenyl-6-(4-(pyridin-2-yl)piperazin-1-yl)pyrimidine-5-carbonitrile;
53. 4-(Methylthio)-2-phenyl-6-(4-(pyridin-2-ylmethyl)piperazin-1-yl)pyrimidine-5-carbonitrile;
54. Ethyl 3-(2-(2-chlorophenyl)-5-cyano-6-(methylthio)pyrimidin-4-ylamino)-4-hydroxy-5-methoxybenzoate;
55. 4-(4-Methoxybenzyl)piperazin-1-yl)-6-(methylthio)-2-phenylpyrimidine-5-carbonitrile; and
56. 4-(5-Amino-3-t-butyl-1H-pyrazol-1-yl)-2-(4-methoxyphenyl)-6-(methylthio)pyrimidine-5-carbonitrile.

According to another feature of the present invention, there is provided a process as shown in the following schemes,

A process for the synthesis of the compounds of the formula I, wherein all the groups are as defined earlier, by reacting the compound of formula G₁ with hydrazine or its derivatives or heterocyclyl group. When the reacting group was hydrazine or its derivatives, the resulting compound (1) was further treated with cyclic anhydrides.

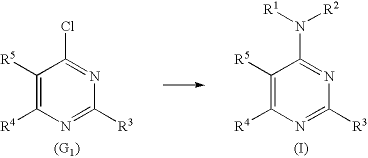

A process for the synthesis of the compounds of the formula (Ia) by reacting the compound of formula G₁ with hydrazine or its derivatives to give G₂. G₂ was further reacted with cyclic anhydrides to give (Ia).

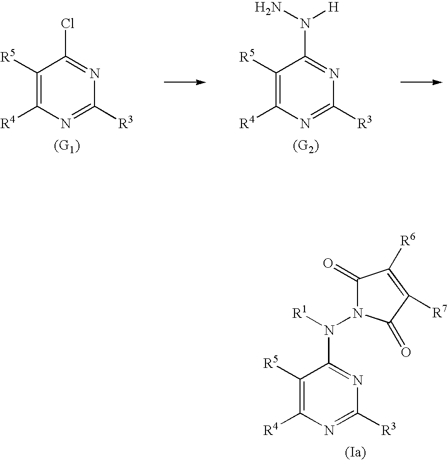

A process for the synthesis of the compounds of the formula (Ib), wherein, R¹ is hydrogen, R² is 3-methyl-1H-pyrrole-2,5-dione and R is selected from halogen; haloalkyl; nitro; hydroxyl; carboxylic acid; ester; amide; alkyl; alkoxy; amino; aminosulfonyl; heterocyclylalkyl; heterocyclylsulfonyl; alkylthio; mercapto; aryl; heteroaryl and heteroarylalkyl groups; which in turn are optionally substituted by halogen; alkyl; alkoxy; aryl and heteroaryl and all the other groups are as defined earlier, is described in the Scheme-1 and Scheme-2, as shown below.

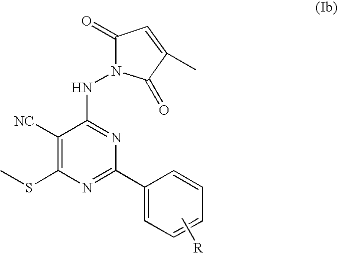

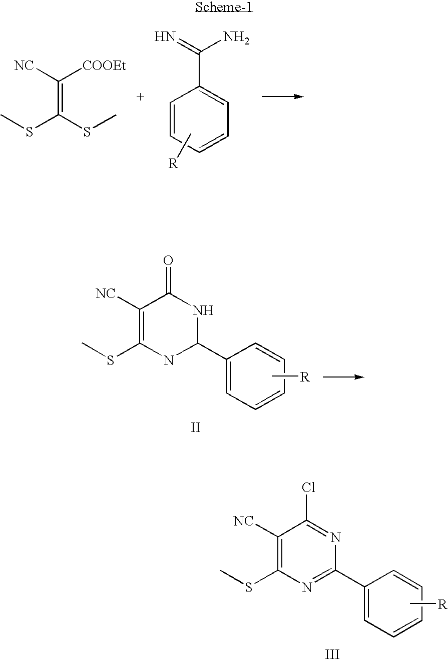

The S,S acetal shown above in the Scheme-1 (prepared according to a literature procedure by Y. Tominaga et al., J Heterocyclic Chem 1988, 25, 959) was treated with substituted or unsubstituted benzamidine in the presence of a base to get the cyclized product II (U.S. Pat. No. 6,107,485/2000 and references cited therein), which when refluxed with POCl₃ furnished III. The reaction of the S,S-acetal with amidines can be carried out in solvents such as THF, acetonitrile, DMF, dioxane, dimethoxyethane and the like in the presence of bases such as sodium hydride, sodium hydroxide, sodium methoxide, sodium ethoxide, potassium tert butoxide, potassium carbonate, cesium carbonate and the like. The reaction was carried out at temperatures ranging from −20° C. to 100° C. for 0-24 h, yields are in the range of 30 to 90%. Chlorination with POCl₃ was performed at temperatures ranging from 0 to reflux for a period ranging from 0-24 h and the yields are in the range of 30 to 90%.

The chloro compound III thus obtained was further transformed to the desired product as shown in the following Scheme-2.

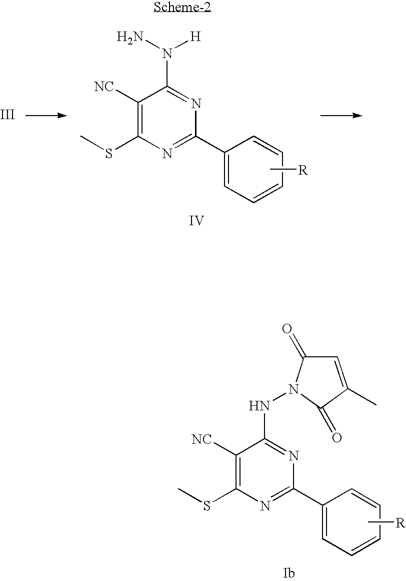

The compound III was treated with hydrazine to provide the hydrazine derivative IV, which when reacted with citraconic anhydride provided the compound (Ib). The reaction of III with hydrazine can be performed in solvents such as acetonitrile, chlorinated solvents such as chloroform and the like, diethylether, dioxane, tetrahydrofuran (THF), dimethylformamide (DMF) and the like at temperatures ranging from −20° C. to reflux for a period ranging from 0-24 h. The reaction of the compound IV with citraconic anhydride can be performed in chlorinated solvents such as chloroform and the like; dioxane, THF, dimethoxyethane, DMF, toluene, hydrocarbon solvents such as hexane and the like at temperatures ranging from 0° C. to reflux.

In yet another embodiment of the present invention, there is provided a process for the preparation of novel heterocyclic compounds of the formula (Ic), shown below, wherein R₃ is methyl and other groups are as defined earlier (Scheme-3).

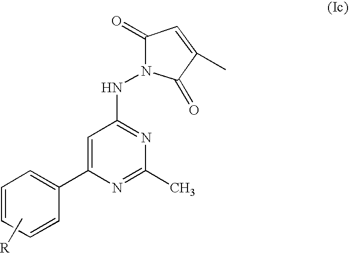

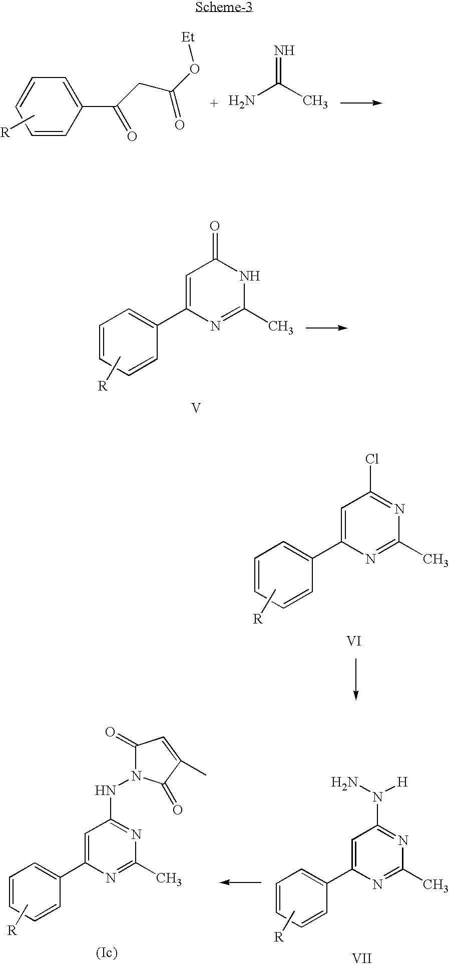

The pyrimidone V is readily prepared from commercially available β-ketoester and acetamidine in the presence of a base. The reaction can be carried out in the presence of bases such as sodium hydroxide, sodium methoxide, sodium ethoxide, potassium t-butoxide, sodium hydride and the like in the presence of solvents such as methanol, ethanol, acetone, acetonitrile, DMF, dioxane, dimethoxyethane and the like, at temperatures ranging from 0° C. to reflux temperatures, for a period ranging from 0 to 24 h. Chlorination with POCl₃ was performed at temperatures ranging from 0° C. to reflux for a period ranging from 0-24 h. The yields are in the range of 30 to 90% to obtain VI. The reaction of VI with hydrazine can be performed in solvents such as acetonitrile, chlorinated solvents such as chloroform and the like; diethyl ether, dioxane, THF, DMF and the like at temperatures ranging from −20° C. to reflux for a period ranging from 0 to 24 h. The reaction of compound VI with citraconic anhydride can be performed in chlorinated solvents such as chloroform and the like, dioxane, THF, dimethoxyethane, DMF, toluene, hydrocarbon solvents such as hexane and the like at temperatures ranging from 0° C. to reflux to obtain (Ic).

Also disclosed is the fact that wherever, benzamidine was used as starting material, it is replaced with substituted benzamidines such as methylbenzamidine, methoxybenzamidine and the like in order to obtain the appropriate substitution in the aromatic ring; wherever β-ketoesters are mentioned in the preparations, substituted β-ketoesters were also used in order to obtain the appropriate substitutions in the aromatic ring.

The pharmaceutically acceptable salts are prepared by reacting the compound of formula (I, Ia) with 1 to 10 equivalents of a base such as sodium hydroxide, sodium methoxide, sodium hydride, potassium t-butoxide, calcium hydroxide, magnesium hydroxide and the like, in solvents like ether, THF, methanol, t-butanol, dioxane, isopropanol, ethanol etc. Mixture of solvents may also be used. Organic bases such as diethanolamine, α-phenylethylamine, benzylamine, piperidine, morpholine, pyridine, hydroxyethylpyrrolidine, hydroxyethylpiperidine, choline, guanidine and the like, ammonium or substituted ammonium salts, aluminum salts. Amino acids such as glycine, alanine, cystine, cysteine, lysine, arginine, phenylalanine etc may be used for the preparation of amino acid salts. Alternatively, acid addition salts wherever applicable are prepared by treatment with acids such as hydrochloric acid, hydrobromic acid, nitric acid, sulfuric acid, phosphoric acid, p-toluenesulphonic acid, methanesulfonic acid, acetic acid, citric acid, maleic acid, salicylic acid, hydroxynaphthoic acid, ascorbic acid, palmitic acid, succinic acid, benzoic acid, benzenesulfonic acid, tartaric acid, oxalic acid and the like in solvents like ethyl acetate, ether, alcohols, acetone, tetrahydrofuran, dioxane etc. Mixture of solvents may also be used.

It should be noted that compounds of the invention may contain groups that may exist in tautomeric forms, and though one form is named, described, displayed and/or claimed herein, all the hydrazine forms are intended to be inherently included in such name, description, display and/or claim.

The stereoisomers of the compounds forming part of this invention may be prepared by using reactants in their single enantiomeric form, in the process wherever possible or by conducting the reaction in the presence of reagents or catalysts in their single enantiomeric form or by resolving the mixture of stereoisomers by conventional methods. Some of the preferred methods include use of microbial resolution, resolving the diastereomeric salts formed with chiral acids such as mandelic acid, camphorsulfonic acid, tartaric acid, lactic acid, and the like wherever applicable or by using chiral bases such as brucine, cinchona alkaloids, their derivatives and the like.

Prodrugs of the compounds of formula (I, Ia) are also contemplated by this invention. A prodrug is an active or inactive compound that is modified chemically through in-vivo physiological action, such as hydrolysis, metabolism and the like, into a compound of this invention following administration of the prodrug to a patient. The suitability and techniques involved in making and using prodrugs are well known by those skilled in the art.

Various polymorphs of the compounds of the general formula (I, Ia), forming part of this invention may be prepared by crystallization of the compounds of formula (I, Ia) under different conditions. For example, using different commonly used solvents, or their mixtures for recrystallization; crystallizations at different temperatures; various modes of cooling, ranging from very fast to very slow cooling during crystallizations. Heating or melting the compounds followed by cooling gradually or immediately, one can also obtain polymorphs. The presence of polymorphs may be determined by solid probe NMR spectroscopy, IR spectroscopy, differential scanning calorimetry and powder X-ray diffraction or other such techniques.

Pharmaceutically acceptable solvates of the compounds of the formula (I, Ia) forming part of this invention may be prepared by conventional methods such as dissolving the compounds of the formula (I, Ia) in solvents such as water, methanol, ethanol, mixture of solvents such as acetone:water, dioxane:water, N,N-dimethylformamide:water and the like, preferably water and recrystallization by using different crystallization techniques

The present invention also provides a pharmaceutical composition, containing one or more of the compounds of the general formula (I, Ia) as defined above, their derivatives, analogs, tautomeric forms, stereoisomers, polymorphs, hydrates, metabolites, prodrugs, pharmaceutically acceptable salts, pharmaceutically acceptable solvates in combination with the usual pharmaceutically employed carriers, diluents and the like.

The pharmaceutical composition may be in the forms normally employed, such as tablets, capsules, powders, syrups, solutions, suspensions and the like, may contain flavorants, sweeteners etc. in suitable solid or liquid carriers or diluents, or in suitable sterile media to form injectable solutions or suspensions. The compositions may be prepared by processes known in the art. The amount of the active ingredient in the composition may be less than 70% by weight. Such compositions typically contain from 1 to 25%, preferably 1 to 15% by weight of active compound, the remainder of the composition being pharmaceutically acceptable carriers, diluents, excipients or solvents.

Suitable pharmaceutically acceptable carriers include solid fillers or diluents and sterile aqueous or organic solutions. The active compound will be present in such pharmaceutical compositions in the amounts sufficient to provide the desired dosage in the range as described above. Thus, for oral administration, the compounds can be combined with a suitable solid or liquid carrier or diluent to form capsules, tablets, powders, syrups, solutions, suspensions and the like. The pharmaceutical compositions, may, if desired, contain additional components such as flavorants, sweeteners, excipients and the like. For parenteral administration, the compounds can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable acid addition salts or alkali or alkaline earth metal salts of the compounds. The injectable solutions prepared in this manner can then be, administered intravenously, intraperitoneally, subcutaneously, or intramuscularly, with intramuscular administration being preferred in humans.

The pharmaceutical compositions of the invention are effective in lowering TNF-α, IL-1, IL-6, IL-1β, IL-8, IL-12 and cyclooxygenases such as COX-1, COX-2 and COX-3 activity without causing ulcers. The pharmaceutical compositions of the invention are thus effective for treating immunological diseases, inflammation, pain disorder, rheumatoid arthritis; osteoporosis; multiple myeloma; uveititis; acute and chronic myelogenous leukemia; ischemic heart disease; atherosclerosis; cancer; ischemic-induced cell damage; pancreatic beta cell destruction; osteoarthritis; rheumatoid spondylitis; gouty arthritis; inflammatory bowel disease; adult respiratory distress syndrome (ARDS); psoriasis; Crohn's disease; allergic rhinitis; ulcerative colitis; anaphylaxis; contact dermatitis; muscle degeneration; cachexia; asthma; bone resorption diseases; ischemia reperfusion injury; brain trauma; multiple sclerosis; sepsis; septic shock; toxic shock syndrome; fever, and myalgias due to infection and diseases mediated by HIV-1; HIV-2; HIV-3; cytomegalovirus (CMV); influenza; adenovirus; the herpes viruses (including HSV-1, HSV-2) and herpes zoster viruses in a mammal.

Generally, the effective dose for treating a particular condition in a patient may be readily determined and adjusted by the physician during treatment to alleviate the symptoms or indications of the condition or disease. Generally, a daily dose of active compound in the range of about 0.01 to 1000 mg/kg of body weight is appropriate for administration to obtain effective results. The daily dose may be administered in a single dose or divided into several doses. In some cases, depending upon the individual response, it may be necessary to deviate upwards or downwards from the initially prescribed daily dose. Typical pharmaceutical preparations normally contain from about 0.2 to about 500 mg of active compound of formula (I, Ia) and/or its pharmaceutically active salts or solvates per dose.

While the compounds of the invention can be administered as the sole active pharmaceutical agent, they can also be used in combination with one or more compounds of the invention or other agents. When administered as a combination, the therapeutic agents can be formulated as separate compositions that are given at the same time or different times, or the therapeutic agents can be given as a single composition.

The term “therapeutically effective amount” or “effective amount” refers to that amount of a compound or mixture of compounds of Formula (I, Ia) that is sufficient to effect treatment, as defined below, when administered alone or in combination with other therapies to a mammal in need of such treatment. More specifically, it is that amount that is sufficient to lower the cytokines such as TNF-α, IL-1, IL-6, IL-1β, IL-8, IL-12 and cyclooxygenases such as COX-1, COX-2 and COX-3.

The term “animal” as used herein is meant to include all mammals, and in particular humans. Such animals are also referred to herein as subjects or patients in need of treatment. The therapeutically effective amount will vary depending upon the subject and disease condition being treated, the weight and age of the subject, the severity of the disease condition, the particular compound of Formula I & Ia, chosen, the dosing regimen to be followed, timing of administration, the manner of administration and the like, all of which can readily be determined by one of ordinary skill in the art.

The term “treatment” or “treating” means any treatment of a disease in a mammal, including:

a) Preventing the disease, that is, causing the clinical symptoms of the disease not to develop;

b) Inhibiting the disease, that is, slowing or arresting the development of clinical symptoms; and/or

c) Relieving the disease, that is, causing the regression of clinical symptoms.

From the foregoing description, one skilled in the art can easily ascertain the essential characteristics of this invention, and without departing from the spirit and scope thereof, make various changes and modifications of the invention to adapt it to various usages and conditions.

The novel heterocyclic compounds of the present invention are useful for the treatment of inflammation and immunological diseases. Particularly the compounds of the present invention are useful for the treatment of cancer, inflammation and immunological diseases those mediated by cytokines such as TNF-α, IL-1, IL-6, IL-1β, IL-8, IL-12, cyclooxygenases such as COX-1, COX-2 and COX-3, lipoxygenases such as 5-LOX, 12-LOX, and 15-LOX, and thromboxane as described in the experimental section providing the biological activity data in various in vitro and in vivo models. The compounds of the present invention are also useful as PDE4 inhibitors, and are useful for treating PDE4 mediated diseases such as asthma, COPD, IBD, arthritis, psoriasis and the like. More particularly, the compounds of the present invention are useful as dual inhibitors of 5-LOX and thromboxane synthase, and are useful for treating lipoxygenase and thromboxane mediated diseases such as asthma, COPD, IBD, arthritis, psoriasis, cancer and the like. Standard literature methods were followed for finding the activity of the compounds in different assay methods. The compounds of the present invention have shown activity better or superior to the compounds disclosed in the earlier literature and hence the novel molecules of the present invention are potentially useful in treating disease conditions as mentioned above.

The present invention is provided by the examples given below, which are provided by the way of illustration only, and should not be considered to limit the scope of the invention. Variation and changes, which are obvious to one skilled in the art, are intended to be within the scope and nature of the invention.

### EXPERIMENTAL PROTOCOLS

### Example 1

### Synthesis of 4-1(3-methyl-2,5-dioxo-2,5-dihydro-1H-pyrrol-1-yl)amino]-6-(methylthio)-2-phenylpyrimidine-5-carbonitrile

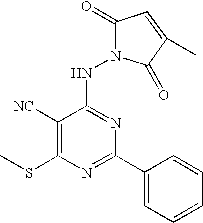

### Step 1: Preparation of the pyrimidinone derivative

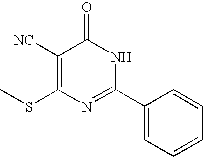

To a stirred solution of benzamidine hydrochloride (5 g, 0.02 mol) in DMF (10 ml) was added DIPEA (4.8 ml, 0.027 mol) and was stirred for 10 min. To the above solution was added ethyl 2-cyano-3,3-bis(methylthio)acrylate (5.5 g, 0.025 mol) and was refluxed at 80° C. for 26 h. The reaction mixture was subsequently filtered, dried; the filtrate was treated with water (150 ml) and dichloromethane (150 ml×3). The combined organic layers were dried over anhydrous sodium sulphate and evaporated to dryness to give the pyrimidone derivative as a solid. Yield 63%, ¹H-NMR (CDCl₃) δ: 2.72 (s, 3H), 7.57-7.61 (t, 2H), 7.67-7.70 (m, 1H), 8.22 (d, 2H), 13.52 (s, 1H); HPLC (purity): 96.9%; Mass calculated for C₁₂H₉N₃OS 243.3, observed 244.1; R_{f}0.52 in chloroform:methanol (9:1).

### Step 2: Preparation of the chloropyrimidine derivative

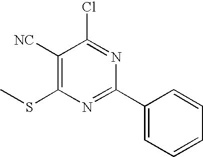

The above made pyrimidone derivative of step 1 (615 mg, 2.53 mmol) was added to POCl₃ (8.2 g, 0.053 mol) and the resulting slurry was refluxed for 24 hours at 80° C. The reaction mixture was cooled to room temperature and was gently poured into ice-cold water (50 ml) to give a precipitate, which was filtered and washed with ice-cold water to furnish the chloropyrimidine derivative. Yield 83.7%; ¹H-NMR (CDCl₃) δ: 2.79 (s, 3H), 7.47-7.56 (m, 3H), 8.46-8.49 (m, 2H); HPLC (purity): 96.9%; Mass calculated for C₁₂H₈ClN₃S 261.7, observed 262.1; R_{f} 0.32 in dichloromethane:methanol (95:5).

### Step 3: Preparation of the hydrazine derivative

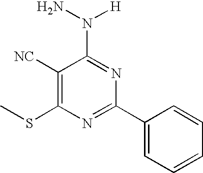

To a solution of the above chloropyrimidone derivative (0.25 g, 0.96 mmoles) in methanol (4 ml) was added hydrazine hydrate (0.24 g, 4.7 mmoles). The resulting solution was stirred at room temperature for 30 minutes. The organic solvent was stripped off at reduced pressure to afford the desired hydrazine as a white solid. Yield 86.7%; ¹H-NMR (CDCl₃) δ: 2.74 (s, 3H), 6.67 (s, 2H), 6.69 (s, 1H), 7.47-7.56 (m, 3H), 8.47 (d, 2H); HPLC (purity): 96.9%; Mass calculated for C₁₂H₁₁N₅S 257.3, observed 258.1; R_{f} 0.32 in dichloromethane:methanol (95:5).

### Step 4: Synthesis of 4-1(3-methyl-2,5-dioxo-2,5-dihydro-1H-pyrrol-1-yl)amino]-6-(methylthio)-2-phenylpyrimidine-5-carbonitrile

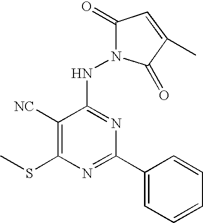

### Synthesis of 4-(3-methyl-2,5-dioxo-2,5-dihydro-1H-pyrrol-1-ylamino)-6-(methylthio)2-phenylpyrimidine-5-carbonitrile

To a solution of the above-mentioned hydrazine derivative (100 mg, 0.39 mmoles) in chloroform (3 ml) was added citraconic anhydride (174 mg, 1.5 mmoles). The resulting solution was stirred at 60° C. for 18 h. Subsequently water (20 ml) and dichloromethane (75 ml) were added to it. The organic layer was separated, dried over anhydrous sodium sulphate and concentrated in vacuum. The resulting residue was subjected to column chromatography using a gradient of ethyl acetate in hexane (0-10%) to yield the desired compound. Yield 27%; ¹H-NMR (CDCl₃) δ: 2.24 (s, 3H), 2.74 (s, 3H), 6.60 (s, 1H), 7.17 (bs, 1H), 7.40-7.43 (m, 2H), 7.48-7.52 (m, 1H), 8.22 (d, 2H); HPLC (purity): 96.5%; Mass calculated for C₁₇H₁₃N₅O₂S 351.4, observed 352.1; R_{f}0.30 in ethyl acetate:hexane (3:7).

Along with the desired compound a by-product was also formed in the above reaction, which was characterized as 4-(4-Methyl-3,6-dioxo-3,6-dihydro pyridazin-1(2H)-yl)-6-(methylthio)-2-phenylpyrimidine-5-carbonitrile based on the spectral data.

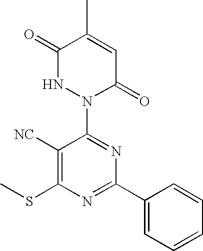

1H-NMR (CDCl₃) δ: 2.26 (s, 3H), 2.75 (s, 3H), 6.65 (s, 1H), 7.48-7.51 (m, 3H), 8.50 (t, 2H), 11.16 (bs, 1H); HPLC (purity): 96.8%; Mass calculated for C₁₇H₁₃N₅O₂S 351.4, observed 352.1; R_{f}0.21 in ethyl acetate:hexane (3:7) as solvent system.

The Following Compounds were Prepared According to the Procedure Given in Example: 1

| S. No | Structure | Analytical data |
|---|---|---|
| 2 | 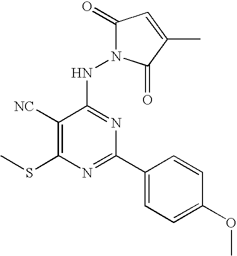 | ¹H-NMR (CDCl₃) δ: 2.16 (s, 3 H), 2.73 (s, 3 H),3.84 (s, 3 H), 6.99 (s, 1 H), 7.06 (d, 2 H), 8.11 (d,2 H), 10.52 (bs, 1 H); HPLC (purity): 90.6%;Mass calculated for C₁₈H₁₅N₅O₃S 381.4, observed382.1; R_{f} 0.62 in ethyl acetate: hexane (1:1). |
| 3 | 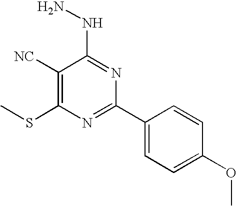 | 1HNMR (CDCl₃) δ: 2.50 (s, 3 H), 3.84 (s, 3 H),7.08 (d, 2 H), 8.40 (d, 2 H); HPLC (purity); HPLC(purity): 96.5%; Mass calculated for C₁₃H₁₃N₅OS−287.3, observed −288.1; R_{f} 0.40 in ethylacetate: hexane (4:6). |
| 4 | 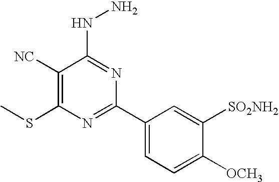 | ¹H-NMR (CDCl₃) δ: 2.67 (s, 3 H), 4.01 (s, 3 H),4.83 (s, 2 H), 7.22 (s, 2 H), 7.35 (d, 1 H), 8.61 (s,1 H), 8.78 (d, 1 H), 9.42 (s, 1 H); HPLC (purity):90.1%; Mass calculated for C₁₃H₁₄N₆O₃S₂−366.4, observed 367.0; R_{f} 0.57 Chloroform:Methanol (9:1). |
| 5 | 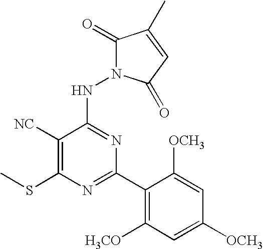 | ¹H-NMR (DMSO) δ: 2.15 (d, 3 H), 2.75 (s, 3 H),3.74-3.79 (m, 3 H), 3.90 (d, 6 H), 7.05 (s, 1 H),7.48 (s, 2 H), 10.89 (s, 1 H); HPLC (purity): 91.3%Mass calculated for C₂₀H₁₉N₅O₅S −441.5,observed 442.1; R_{f} 0.41 Ethyl acetate: Hexanes(1:1). |
| 6 | 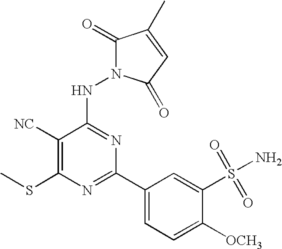 | ¹HNMR (DMSO) δ: 2.16 (s, 3 H), 2.74 (s, 3 H),3.98 (s, 3 H), 6.93 (s, 1 H), 7.21 (s, 2 H), 7.36 (d,1 H), 8.43 (d, 1 H), 8.53 (s, 1 H), 10.83 (s, 1 H):HPLC purity): 94%; Mass calculated forC₁₈H₁₆N₆O₅S₂ −460.5, observed 461.0; R_{f} 0.4Chloroform: Methanol (9.5:0.5). |
| 7 | 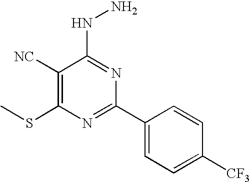 | ¹H-NMR (DMSO) δ: 2.69 (s, 3 H), 7.90 (d, 2 H),8.63 (d, 2 H); HPLC (purity): 97.3%; Masscalculated for C₁₃H₁₀F₃N₅S −325.3, observed326.0; R_{f} 0.72 (Ethyl acetate: Hexanes (1:1). |
| 8 | 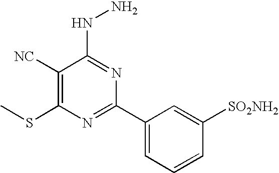 | ¹H-NMR (DMSO) δ: 2.70 (s, 3 H), 4.89 (s, 2 H),7.51 (s, 2 H), 7.72-7.76 (m, 1 H), 8.00 (d, 1 H),8.63 (s, 1 H), 8.84 (s, 1 H), 9.52 (s, 1 H); HPLC(purity): 93%; Mass calculated for C₁₂H₁₂N₆O₂S₂−336.4, observed 377.0; R_{f} 0.38(Chloroform: Methanol (9.5:0.5). |
| 9 | 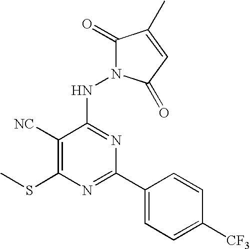 | ¹H-NMR (CDCl₃) δ: 2.25-2.28 (m, 3 H), 2.72 (s,3 H), 6.67 (d, 1 H), 7.26 (s, 1 H), 7.73 (d, 2 H), 8.56(d, 2 H); HPLC (purity): 93.6%; Mass calculatedfor C₁₈H₁₂F₃N₅O₂S −419.4, observed 420.0; R_{f}0.73 (Ethyl acetate: Hexanes (1:1). |
| 10 | 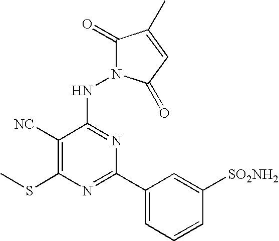 | ¹H-NMR (DMSO) δ: 2.09-2.17 (s, 3 H), 2.77 (s,3 H), 6.95 (s, 1 H), 7.51 (s, 2 H), 7.72-7.76 (m,1 H), 8.02 (d, 1 H), 8.41 (d, 1 H), 8.54 (s, 1 H),10.94 (s, 1 H); HPLC (purity): 92.2%; Masscalculated for C₁₇H₁₄N₆O₄S₂ −430.5, observed431.1; R_{f} 0.45 (Chloroform: Methanol (9:1). |
| 11 | 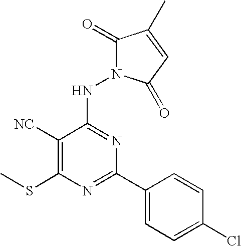 | ¹H-NMR (CDCl₃) δ: 2.24 (d, 3 H), 2.73 (s, 3 H),6.61 (d, 1 H), 7.08-7.12 (d, 2 H), 7.19 (s, 1 H), 8.24(d, 2 H); HPLC (purity): 90.8%; Mass calculatedfor C₁₇H₁₂ClN₅O₂S −385.8, observed 386.1; R_{f}0.79 (Ethyl acetate: Hexane (1:1). |
| 12 | 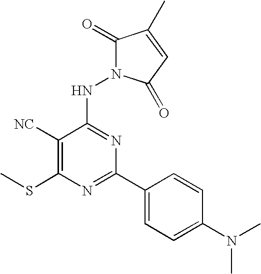 | ¹H-NMR (CDCl₃) 3: 2.23 (d, 3 H), 2.70 (s, 3 H),3.06 (s, 6 H), 6.58 (d, 1 H), 6.65 (d, 2 H), 8.12 (d,2 H); HPLC (purity): 97.5%; Mass calculated forC₁₉H₁₈N₆O₂S −394.5, observed 395.1; R_{f} 0.68(Ethyl acetate: Hexane (1:1). |
| 13 | 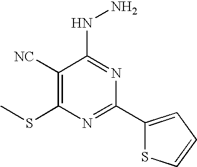 | ¹H-NMR (DMSO) δ: 2.62 (s, 3 H), 7.21-7.23 (m,1 H), 7.84 (d, 1 H), 8.01 (d, 1 H); HPLC (purity):97.9%; Mass calculated for C₁₀H₉N₅S −263.3,observed 304.1; R_{f} 0.8 (Ethyl acetate: Hexane(1:1). |
| 14 | 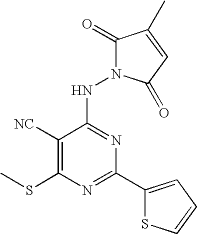 | ¹H-NMR (CDCl₃) δ: 2.24 (s, 3 H), 2.69 (s, 3 H),6.59 (s, 1 H), 7.09-7.11 (m, 1 H), 7.27 (d, 1 H),7.52 (d, 1 H), 7.85 (s, 1 H); HPLC (purity): 94.7%;Mass calculated for C₁₅H₁₁N₅O₂S₂ −357.4,observed 358.1; R_{f} 0.72 (Ethyl acetate: Hexane(1:1). |
| 15 | 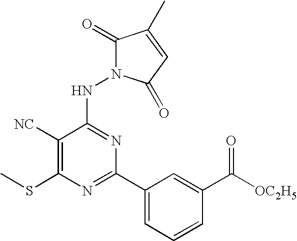 | ¹H-NMR (CDCl₃) δ: 1.40-1.44 (m, 3 H), 2.27 (d,3 H), 2.76 (s, 3 H), 4.38-4.43 (m, 2 H), 6.64 (s,1 H), 7.14 (s, 1 H), 7.49-7.53 (m, 1 H), 8.17 (d,1 H), 8.46 (d, 1 H), 8.85 (s, 1 H); HPLC (purity):91.4%; Mass calculated for C₂₀H₁₇N₅O₄S−423.4, observed 424.1; R_{f} 0.58Ethyl acetate: Hexane (4:6). |
| 16 | 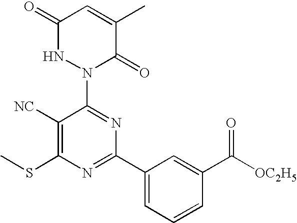 | ¹H-NMR (DMSO) δ: 1.36-1.39 (m, 3 H), 2.18 (s,3 H), 2.78 (s, 3 H), 4.36-4.42 (m, 2 H), 7.07 (s,1 H), 7.72-7.76 (m, 1 H), 8.15 (d, 1 H), 8.78 (d,1 H), 9.09 (s, 1 H), 14.58 (s, 1 H); HPLC (purity):92.6%; Mass calculated for C₂₀H₁₇N₅O₄S−423.4, observed 424.1; R_{f} 0.51Ethyl acetate: Hexane (4:6). |
| 17 | 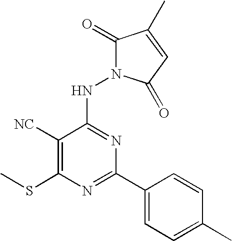 | ¹H-NMR (DMSO) δ: 2.39 (d, 3 H), 2.74 (s, 3 H),3.19 (s, 3 H), 7.06 (s, 1 H), 7.37 (d, 2 H), 8.40 (d,2 H), 14.42 (s, 1 H); HPLC (purity): 95.8%; Masscalculated for C₁₈H₁₅N₅O₂S −365.4, observed366.1; R_{f} 0.32 Ethyl acetate: Hexane (3:7). |
| 18 | 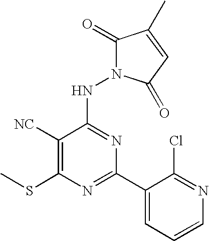 | ¹H-NMR (CDCl₃) δ: 2.18 (d, 3 H), 2.69 (s, 3 H),6.52 (d, 1 H), 7.33-7.36 (m, 1 H), 7.39-7.52 (br, s,1 H), 8.15 (d, 1 H), 8.48 (d, 1 H); HPLC (purity):92.7%; Mass calculated for C₁₆H₁₁ClN₆O₂S−386.8, observed 387.0; R_{f} 0.46(Ethyl acetate: Hexane (1:1). |
| 19 | 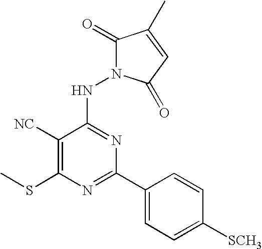 | ¹H-NMR (DMSO) δ: 2.16 (s, 3 H), 2.52 (s, 3 H),2.77 (s, 3 H), 6.99 (s, 1 H), 7.36 (d, 2 H), 8.06 (d,2 H), 10.76 (s, 1 H); HPLC (purity): 95.2%; Masscalculated for C₁₈H₁₅N₅O₂S −397.5, observed398.0; R_{f} 0.32 (Ethyl acetate: Hexane (3:7). |
| 20 | 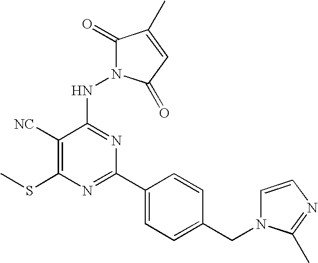 | ¹H-NMR (CDCl₃) δ: 2.23 (d, 3 H), 2.40 (s, 3 H),2.73 (s, 3 H), 5.14 (s, 2 H), 6.60 (d, 2 H), 6.88 (s,1 H), 7.06-7.08 (m, 4H), 8.22 (s, 1 H); HPLC(purity): 92.4%; Mass calculated forC₂₂H₁₉N₇O₂S −445.5, observed 446.1; R_{f} 0.56(Chloroform: Methanol (9:1). |
| 21 | 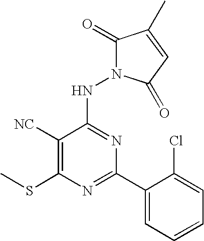 | ¹H-NMR (CDCl₃) δ: 2.17 (s, 3 H), 2.68 (s, 3 H),6.51 (s, 1 H), 7.30-7.34 (m, 1 H), 7.36-7.39 (m,1 H), 7.43 (d, 1 H), 7.79 (d, 1 H); HPLC (purity):97.7%; Mass calculated for C₁₇H₁₂ClN₅O₂S−385.8, observed 386.0; R_{f} 0.67(Ethyl acetate: Hexane (1:1). |
| 22 | 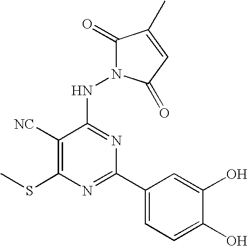 | ¹H-NMR (DMSO) δ: 2.15 (s, 3 H), 2.71 (s, 3 H),6.68 (s, 1 H), 7.12 (s, 1 H), 7.59 (d, 2 H), 9.22 (s,1 H), 9.81 (s, 1 H) 10.65 (s, 1 H); HPLC (purity):95.6%; Mass calculated for C₁₇H₁₃N₅O₄S−383.4, observed 384.0; R_{f} 0.35(Chloroform: Methanol (9:1). |
| 23 | 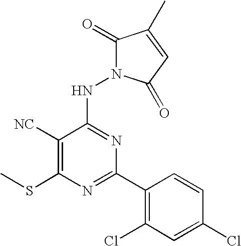 | ¹H-NMR (DMSO) δ: 2.08 (s, 3 H), 2.68 (s, 3 H),6.89 (s, 1 H), 7.57 (d, 1 H), 7.73 (s, 1 H), 7.85 (d,1 H), 10.88 (s, 1 H); HPLC (purity): 97.2%; Masscalculated for C₁₇H₁₁Cl₂N₅O₂S −420.3, observed420.0; R_{f} 0.4 (Ethyl acetate: Hexane (3:7). |
| 24 | 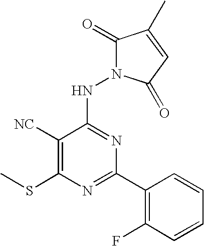 | ¹H-NMR (CDCl₃) δ: 2.21 (s, 3 H), 2.70 (s, 3 H),6.55 (s, 1 H), 7.07-7.12 (m, 1 H), 7.15-7.22 (m,1 H), 7.46 (d, 1 H), 8.08 (d, 1 H); HPLC (purity):83.4%; Mass calculated for C₁₇H₁₂FN₅O₂S−369.4, observed 370.0; R_{f} 0.33(Ethyl acetate: Hexane (3:7). |
| 25 | 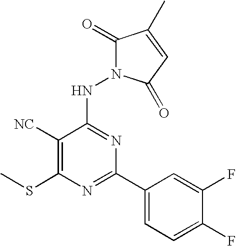 | ¹H-NMR (DMSO) δ: 2.17 (s, 3 H), 2.76 (s, 3 H),6.72 (s, 1 H), 7.06 (s, 1 H), 7.64 (d, 1 H), 8.39 (d,1 H), 14.54 (s, 1 H); HPLC (purity): 95.3%; Masscalculated for C₁₇H₁₁F₂N₅O₂S −387.4, observed388.0; R_{f} 0.67 (Ethyl acetate: Hexane (1:1). |
| 26 | 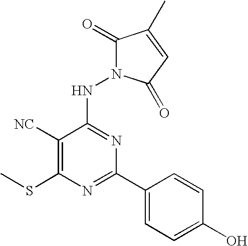 | ¹H-NMR (DMSO) δ: 2.16 (s, 3 H), 2.72 (s, 3 H),6.92 (s, 1 H), 7.05 (d, 2 H), 8.36 (d, 2 H), 10.09 (s,1 H), 14.29 (s, 1 H); HPLC (purity): 99.8%; Masscalculated for C₁₇H₁₃N₅O₃S −367.4, observed368.1; R_{f} 0.54 (Chloroform: Methanol (9:1). |
| 27 | 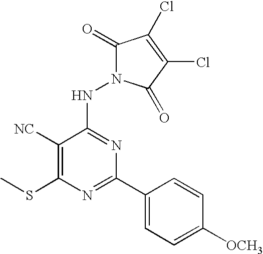 | ¹H-NMR (CDCl₃) δ: 2.76 (s, 3 H), 3.87 (s, 3 H),7.08 (d, 2 H), 8.18 (d, 2 H), 11.15 (s, 1 H); HPLC(purity): 92.4%; Mass calculated forC₁₇H₁₁Cl₂N₅O₃S −436.3, observed 436.0; R_{f} 0.57(Ethyl acetate: Hexane (3:7). |
| 28 | 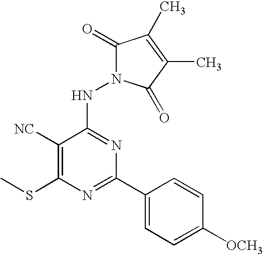 | ¹H-NMR (CDCl₃) δ: 2.11 (s, 6 H), 2.72 (s, 3 H),3.87 (s, 3 H), 6.91 (d, 2 H), 7.12 (s, 1 H), 8.18 (d,2 H); HPLC (purity): 82.2%; Mass calculated forC₁₉H₁₇N₅O₃S −395.4, observed 396.1; R_{f} 0.37(Ethyl acetate: Hexane (3:7). |
| 29 | 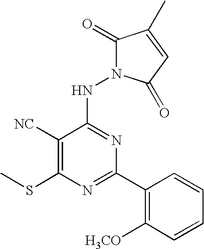 | ¹H-NMR (CDCl₃) δ: 2.17 (s, 3 H), 2.67 (s, 3 H),3.81 (s, 3 H), 6.50 (s, 1 H), 6.96-7.01 (m, 2 H),7.41-7.45 (m, 1 H), 7.75 (d, 1 H); HPLC (purity):98.9%; Mass calculated for C₁₈H₁₅N₅O₃S−381.4, observed 382.1; R_{f} 0.44(Ethyl acetate: Hexane (1:1). |
| 30 | 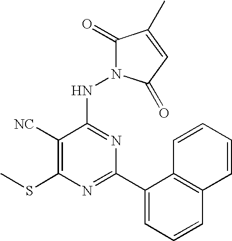 | ¹H-NMR (CDCl₃) δ: 2.12 (s, 3 H), 2.71 (s, 3 H),6.45 (s, 1 H), 7.30 (d, 1 H), 7.47-7.55 (m, 3 H),7.88 (d, 1 H), 7.98 (d, 1 H), 8.06 (d, 1 H), 8.55 (s,1 H); HPLC (purity): 99.4%; Mass calculated forC₂₁H₁₅N₅O₂S −401.4, observed 402.1; R_{f} 0.49(Ethyl acetate: Hexane (3:7). |
| 31 | 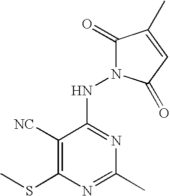 | ¹H-NMR (CDCl₃) δ: 2.19 (d, 3 H), 2.46 (s, 3 H),2.60 (s, 3 H), 6.51-6.52 (m, 1 H), 7.04 (s, 1 H);HPLC (purity): 97.1%; Mass calculated forC₁₂H₁₁N₅O₂S −289.3, observed 290.0; R_{f} 0.44(Ethyl acetate: Hexane (3:7). |
| 32 | 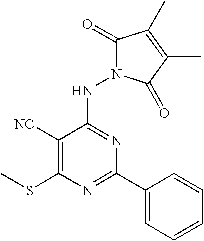 | ¹H-NMR (CDCl₃) δ: 2.12 (s, 6 H), 2.74 (s, 3 H),7.20 (s, 1 H), 7.39-7.43 (m, 2 H), 7.48-7.52 (m,1 H), 8.21 (d, 2 H); HPLC (purity): 99.4%; Masscalculated for C₁₈H₁₅N₅O₂S −365.4, observed366.1; R_{f} 0.57 (Ethyl acetate: Hexane (3:7). |
| 33 | 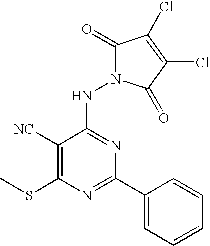 | ¹H-NMR (DMSO) δ: 2.75 (s, 3 H), 7.54 (d, 2 H),7.59-7.62 (m, 1 H), 8.16-8.18 (m, 2 H), 11.32 (s,1 H); HPLC (purity): 89.05%; Mass calculatedfor C₁₆H₉ClN₅O₂S −406.3, observed 406.0; R_{f}0.65 (Ethyl acetate: Hexane (3:7). |
| 34 | 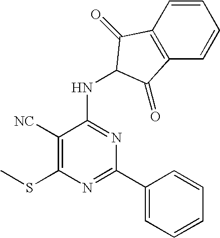 | ¹H-NMR (CDCl₃) δ: 2.75 (s, 3 H), 7.41-7.45 (m,3 H), 7.52 (s, 1 H), 7.88-7.90 (m, 2 H), 8.01-8.03(m, 2 H), 8.12 (d, 2 H); HPLC (purity): 99.6%;Mass calculated for C₂₀H₁₃N₅O₂S −387.4,observed 388.0; R_{f} 0.51 (Ethyl acetate: Hexane(3:7). |
| 35 | 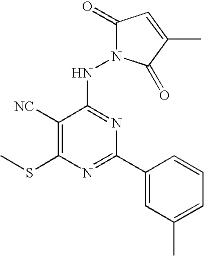 | ¹H-NMR (CDCl₃) δ: 2.24 (d, 3 H), 2.39 (s, 3 H),2.75 (s, 3 H), 6.61 (d, 1 H), 7.14 (s, 1 H), 7.32 (d,3 H), 8.03 (s, 1 H); HPLC (purity): 99%; Masscalculated for C₁₈H₁₅N₅O₂S −365.4, observed365.9; R_{f} 0.55 (Ethyl acetate: Hexane (3:7). |
| 36 | 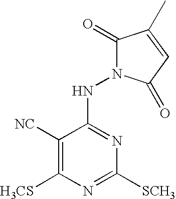 | ¹H-NMR (CDCl₃) δ: 2.18 (d, 3 H), 2.42 (s, 3 H),2.59 (s, 3 H), 6.53 (d, 1 H), 7.25 (s, 1 H); HPLC(purity): 98.4%; Mass calculated forC₁₂H₁₁N₅O₂S −321.4, observed 322.0; R_{f} 0.34(Ethyl acetate: Hexane (3:7). |

### Example 37

### Synthesis of 2-methyl-5-{2-methyl-6-[(3-methyl-2,5-dioxo-2,5-dihydro-1H-pyrrol-1yl)amino]pyrimidin-4-yl benzenesulphonamide

### Step 1:Preparation of the pyrimidone derivative

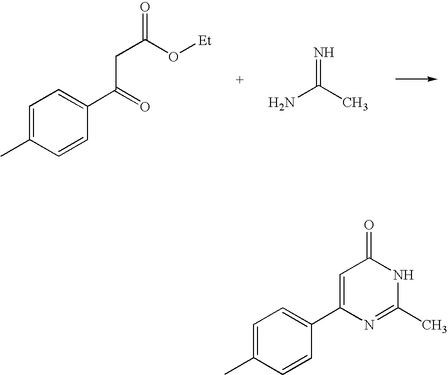

To a solution of NaOEt (17.3 g, 0.254 mol) in 50 ml of ethanol, diketoester shown above (15 g, 0.72 mol) and acetamidine hydrochloride (12 g, 0.13 mol) were added and the reaction mixture was refluxed at 90° C. for 16 h. Subsequently the solvent was distilled off and water (200 ml) was added to the crude mixture to obtain an off white colored solid, which was filtered and washed with hexane (200 ml), dried under high vacuum to afford the required product. Yield 62.5%; ¹H-NMR (CDCl₃) δ: 2.41 (s, 3H), 2.59 (s, 3H), 6.745 (s, 1H), 7.27-7.28 (m, 2H), 7.87-7.89 (m, 2H), 13.38 (s, 1H (D₂O exchangeable proton); Mass calculated for C₁₂H₁₂N₂O 200.20, observed 201.1; HPLC (purity) 99.4%; R_{f} 0.25 in ethyl acetate:hexane (5:4).

### Step 2: Preparation of 4-chloro-2-methyl-6-(4-methylphenyl)pyrimidine

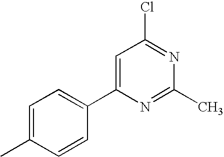

To a cold solution (5° C.) of POCl₃ (22.9 g, 0.150 mol) was added 2-methyl-6-(4-methylphenyl)pyrimidin-4(3H)-one (3 g, 0.015 mol) made above and the resulting mixture was refluxed at 100° C. for 3 h. Subsequently the reaction mixture was poured into crushed ice and sodium bicarbonate (3 g) was added to it. The organic layer was extracted with ethyl acetate (250 ml), dried over anhyd sodium sulphate and then concentrated to afford the crude compound (2.675 g, 82% yield). ¹H-NMR (CDCl₃) δ: 2.52 (s, 3H), 2.72 (s, 3H), 7.49-7.55 (m, 3H), 8.04-8.06 (m, 2H); Mass calculated for C₁₂H₁₁ClN₂ 218.60, observed 219.1; HPLC (purity) 88.4%; R_{f} 0.85 in ethyl acetate:hexane (1:1).

### Step 3: Preparation of 5-(6-chloro-2-methylpyrimidin-4-yl)-2-methylbenzene sulphonamide

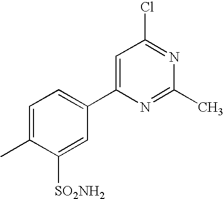

To a cold solution (5° C.) of chlorosulphonic acid (23.9 g, 0.206 mol) was added the above made chloropyrimidine derivative (1.5 g, 0.0068 mol) and the resulting mixture stirred at an ambient temperature for 15 min. It was then brought to room temperature and stirred continuously for 48 h. Subsequently the reaction mixture was poured into crushed ice and NaHCO₃ (3 g) was added to it. Ethyl acetate (200 ml) was added and the organic layer was separated, washed with brine, dried over anhydrous sodium sulphate and then concentrated to give the crude solid (0.93 g, 44% yield). The crude solid was dissolved in dichloromethane (30 ml) and cooled to 5° C. Ammonia gas was purged into this solution at an ambient temperature for 20 min until a solid was thrown out. The resulting mixture was poured into crushed ice and extracted with ethyl acetate (100 ml). The organic layer was separated, washed with brine, dried over anhydrous sodium sulphate and then concentrated to afford the title compound as a solid (0.72 g, 85% yield). ¹H-NMR (DMSO) δ: 2.66-2.69 (m, 6H), 7.54-7.58 [(m, 2H), D₂O exchangeable protons], 7.58-7.60 (d, 1H); 8.145 (s, 1H), 8.31-8.33 (d, 1H), 8.734 (s, 1H); Mass calculated for C₁₂H₁₂ClN₃O₂S 297.7, observed 298.1; HPLC (purity) 98.3%; R^{f}0.85 in ethyl acetate:hexane (3:7).

### Step 4: Preparation of 5-(6-hydrazino-2-methylpyrimidin-4-yl)-2-methyl benzenesulphonamide

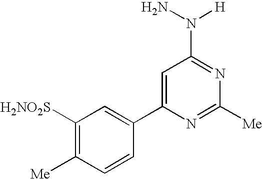

To a solution of the chlorosulfonamide derivative made above (0.25 g, 0.84 mmol) in THF (7 ml) was added hydrazine hydrate (0.4378 g, 0.0084 moles) and the resulting mixture was refluxed at 70° C. for 6 h. Subsequently the solvent was evaporated and the crude mass obtained was washed first with water (20 ml) and then with hexane (25 ml). The solid obtained was dried under high vacuum to afford the hydrazine derivative as an off white solid. Yield 49%; ¹H-NMR (DMSO) δ: 2.60 (s, 3H), 2.68 (s, 3H), 7.58-7.60 (m, 2H), D₂O exchangeable protons), 7.62 (s, 1H) 7.89 (s, 1H) D₂O exchangeable proton), 8.15 (s, 1H), 8.33-8.36 (d, 1H), 8.75 (s, 1H); Mass calculated for C₁₂H₁₅NSO₂S 293.3, observed 294.1; R_{f} 0.20 in ethyl acetate:hexane (1:1).

### Step 5: Synthesis of 2-methyl-5-{2-methyl-6-[(3-methyl-2,5-dioxo-2,5-dihydro-1H-pyrrol-1yl)amino]pyrimidin-4-yl benzenesulphonamide

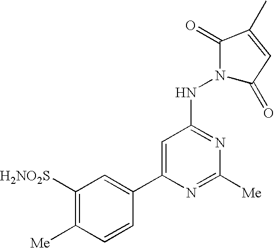

To a solution of the hydrazine derivative made above (0.1 g, 0.34 mmol) in a mixture of chloroform (10 ml) and methanol (1 ml) was added citraconic anhydride (0.191 g, 17 mmol) and the resulting mixture was refluxed at 80° C. for 4 hours. The reaction mixture was poured into crushed ice and extracted with ethyl acetate (300 ml). The organic layer was separated, dried over anhydrous sodium sulphate and then concentrated. The crude mass obtained was purified by column chromatography to afford the compound as a pale yellow solid. ¹H-NMR (DMSO) δ: 2.10 (s, 3H), 2.52 (s, 3H), 2.59 (s, 3H), 6.854-6.856 (d, 1H), 7.0527 (s, 1H), 7.504 (m, 2H (D₂O exchangeable protons)), 7.534 (s, 1H), 8.139-8.15 (d, 1H), 8.597 (s, 1H), 9.841-[(s, 1H) (D₂O exchangeable proton)]; Mass calculated for C₁₇H₁₇N₅O₄S—387.41, observed 388.1; HPLC (purity) 85.7%; R_{f}: 0.65 in 100% ethyl acetate.

The following compound was prepared according to the procedure given in Example: 37

| | | |
|---|---|---|
| 38 | 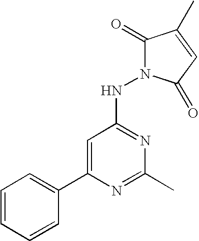 | ¹H-NMR (CDCl₃) δ: 2.21 (s, 3 H), 2.59 (s, 3 H),6.53 (d, 1 H), 6.67 (s, 1 H), 7.45-7.47 (m, 3 H),7.89-7.91 (m, 2 H); HPLC (purity): 96.8%;Mass calculated for C₁₆H₁₄N₄O₂ 294.3,observed 295.1; R_{f} 0.45 inethyl acetate: hexane (1:1). |
| 39 | 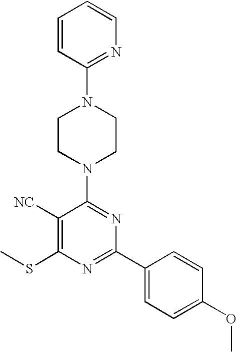 | ¹H-NMR (CDCl₃) δ: 2.71 (s, 3 H), 3.73-3.75(m, 4 H), 3.89 (s, 3 H), 4.17-4.20 (m, 4 H),6.65-6.70 (m, 2 H), 6.98 (m, 2 H), 7.51-7.55(m, 1 H), 8.22 (s, 1 H), 8.39 (d, 2 H); HPLC(purity): 98.4%; Mass calculated forC₂₂H₂₂N₆OS −418.55, observed −419.1; R_{f}0.51 in ethyl acetate: hexane (3:7). |
| 40 | 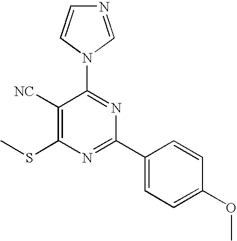 | 1H-NMR (CDCl₃) δ: 2.82 (s, 3 H), 3.88 (s,3 H), 7.14 (d, 2 H), 7.25 (s, 1 H), 8.09 (s, 1 H),8.52 (d, 2 H), 8.71 (s, 1 H); HPLC (purity);HPLC (purity): 99.5%; Mass calculated forC₁₆H₁₃N₅OS 323.4, observed 324.1; R_{f} 0.57in ethyl acetate: hexane (1:1). |
| 41 | 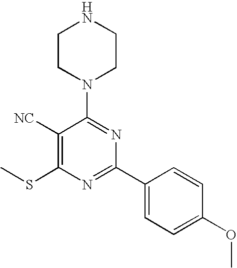 | 1HNMR (CDCl₃) δ: 2.70 (s, 3 H), 3.00-3.02(m, 4 H), 3.88 (s, 3 H), 4.00-4.02 (m, 4 H),6.95-7.00 (m, 2 H), 8.36-8.41 (m, 2 H); HPLC(purity): 99.8%; Mass calculated forC₁₇H₁₉N₅OS 341.4, observed 342.1; R_{f} 0.27in chlorofrom: methanol (95:5). |
| 42 | 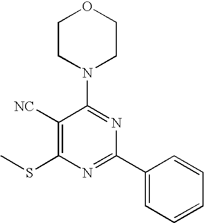 | 1HNMR (CDCl₃) δ: 2.73 (s, 3 H), 3.82-3.85(m, 4 H), 4.06-4.08 (m, 4 H), 7.45-7.54 (m,3 H), 8.40 (t, 2 H); HPLC (purity); HPLC(purity): 99.4%; Mass calculated forC₁₆H₁₆N₄OS 312.4, observed 313.1; R_{f} 0.30in ethyl acetate: hexane (1:9) |
| 43 | 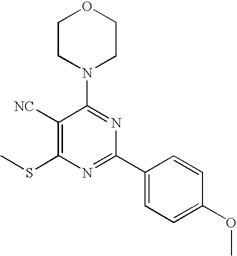 | 1H-NMR (CDCl₃) δ: 2.70 (s, 3 H), 3.81-3.87(m, 4 H), 3.90 (s, 3 H), 4.03-4.05 (m, 4 H),6.95-7.00 (m, 2 H), 8.36-8.38 (m, 2 H); HPLC(purity): 99.0%; Mass calculated forC₁₇H₁₈N₄O₂S −342.4, observed −343.1; R_{f}0.55 in ethyl acetate: hexane (3:7). |
| 44 | 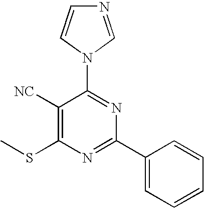 | ¹H-NMR (CDCl₃) δ: 2.84 (s, 3 H), 7.29 (s,1 H), 7.53-7.64 (m, 3 H), 8.14 (s, 1 H), 8.52 (d,2 H), 8.82 (s, 1 H); HPLC (purity): 98.5%;Mass calculated for C₁₅H₁₁N₅S −293.3,observed −294.0; R_{f} 0.25 inethyl acetate: hexane (2:8). |
| 45 | 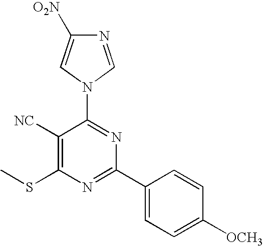 | ¹H-NMR (CDCl₃) δ: 2.85 (s, 3 H), 3.94 (s,3 H), 7.06 (d, 2 H), 8.48 (d, 2 H), 8.67 (d, 1 H),8.84 (d, 1 H); HPLC (purity): 91.9%; Masscalculated for C₁₆H₁₂N₆O₃S −368.4,observed 369.0; R_{f} 0.47(Ethyl acetate: Hexanes (3:7). |
| 46 | 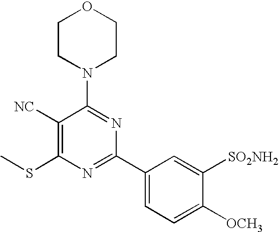 | ¹H-NMR (DMSO) δ: 2.69 (s, 3 H), 3.75 (s,3 H), 4.00 (m, 8 H), 7.27 (s, 2 H), 7.35 (d, 1 H),8.59 (s, 1 H), 8.77 (d, 1 H); HPLC (purity):96.2%; Mass calculated for C₁₇H₁₉N₅O₄S₂−421.5, observed 422.1; R_{f} 0.68(Chloroform: Methanol (9:1). |
| 47 | 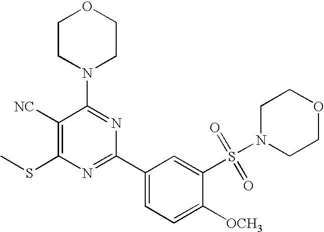 | ¹H-NMR (CDCl₃) δ: 2.50 (d, 4 H), 2.69 (s,3 H), 3.11 (d, 4 H), 3.60 (d, 4 H), 3.74-3.76 (m,4 H), 3.98 (s, 3 H), 7.42 (d, 1 H), 8.61-8.64 (m,1 H), 8.74 (d, 1 H); HPLC (purity): 95.3%;Mass calculated for C₂₁H₂₅N₅O₅S₂ −491.6,observed 492.0; R_{f} 0.25(Ethyl acetate: Hexanes (1:1). |
| 48 | 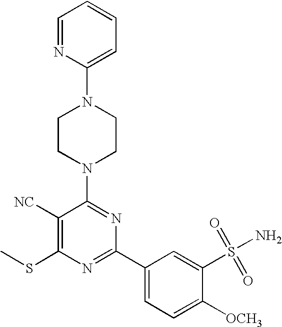 | ¹H-NMR (DMSO) δ: 2.70 (s, 3 H), 3.72 (s,4 H), 4.00 (s, 3 H), 4.11 (s, 4 H), 6.67-6.70 (m,1 H), 6.87 (d, 1 H), 7.28 (s, 2 H), 7.37 (d, 1 H),7.56-7.59 (m, 1 H), 8.14 (d, 1 H), 8.60 (d, 1 H),8.81 (d, 1 H); HPLC (purity): 91.1%; Masscalculated for C₂₂H₂₃N₇O₃S₂ −497.6,observed 498.1; R_{f} 0.57(Chloroform: Methanol (9.5:0.5). |
| 49 | 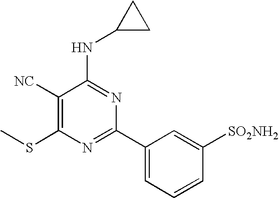 | ¹H-NMR (DMSO) δ: 0.78-0.81 (m, 2 H),0.82-0.85 (m, 2 H), 2.72 (s, 3 H), 3.08 (m,1 H), 7.52 (s, 2 H), 7.73-7.77 (m, 1 H), 8.02 (d,1 H) 8.28 (s, 1 H), 8.62 (d, 1 H), 8.85 (s, 1 H);HPLC (purity): 99.7%; Mass calculated forC₁₅H₁₅N₅O₂S₂ −361.4 observed 362.1; R_{f}0.75 (Ethyl acetate: Hexane (7:3). |
| 50 | 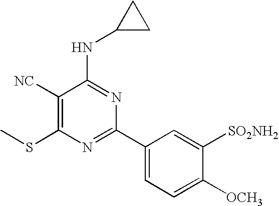 | ¹H-NMR (DMSO) δ: 0.71-0.76 (m, 2 H),0.80-0.83 (m, 2 H), 2.69 (s, 3 H) 3.00 (m, 1 H),4.00 (s, 3 H), 7.22 (s, 2 H), 7.36 (d, 1 H), 8.20(s, 1 H), 8.61 (d, 1 H), 8.83 (s, 1 H); HPLC(purity): 95.3%; Mass calculated forC₁₆H₁₇N₅O₃S₂ −391.5, observed 392.1; R_{f}0.5 (Ethyl acetate: Hexane (7:3). |
| 51 | 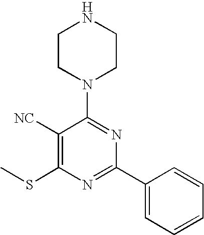 | ¹H-NMR (DMSO) δ: 2.70 (s, 3 H), 2.84-2.86(m, 4 H), 3.92-3.94 (m, 4 H), 7.52-7.60 (m,3 H), 8.39 (d, 2 H); HPLC (purity): 95.6%;Mass calculated for C₁₆H₁₇N₅S −311.4,observed 312.1; R_{f} 0.47(Chloroform: Methanol (9:1). |
| 52 | 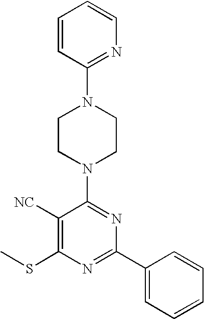 | ¹H-NMR (CDCl₃) δ: 2.74 (s, 3 H), 3.74-3.77(m, 4 H), 4.17-4.22 (m, 4 H), 6.66-6.70 (m,2 H), 7.47-7.99 (m, 3 H), 8.22 (d, 2 H), 8.43 (d,2 H); HPLC (purity): 98%; Mass calculatedfor C₂₁H₂₀N₆S −388.5, observed 389.1; R_{f}0.66 (Ethyl acetate: Hexane (3:7). |
| 53 | 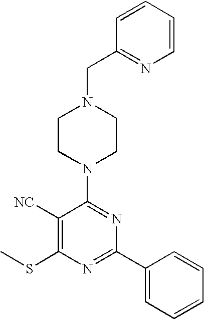 | ¹H-NMR (CDCl₃) δ: 2.68 (s, 4 H), 2.72 (s,3 H), 3.74 (s, 2 H), 4.27 (s, 4 H), 7.19-7.22 (m,1 H), 7.44-7.52 (m, 4 H), 7.67-7.71 (m, 1 H),8.40 (d, 2 H), 8.60 (d, 1 H); HPLC (purity):94.6%; Mass calculated for C₂₂H₂₂N₆S−402.5, observed 403.1; R_{f} 0.35(Ethyl acetate: Hexane (1:1). |
| 54 | 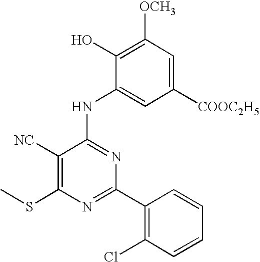 | ¹H-NMR (CDCl₃) δ: 1.32-1.44 (m, 3 H), 2.71(s, 3 H), 3.96 (s, 3 H), 4.30-4.38 (m, 2 H), 7.38-7.42(m, 3 H), 7.49-7.51 (m, 1 H), 7.71 (s,1 H), 7.96-7.98 (s, 1 H), 8.78 (s, 1 H); HPLC(purity): 85.7%; Mass calculated forC₂₂H₁₉ClN₄O₄S −470.9, observed 471.0; R_{f}0.62 (Ethyl acetate: Hexane (1:1). |
| 55 | 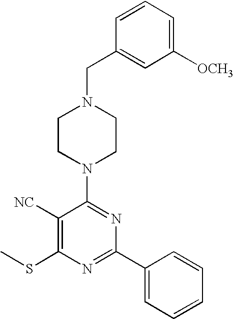 | ^{1 H-NMR (CDCl}₃) δ: 2.58-2.60 (m, 4 H), 2.72(s, 3 H), 3.54 (s, 2 H), 3.83 (s, 3 H), 4.07-4.09(m, 4 H), 6.83 (s, 1 H), 6.92 (d, 2 H), 7.23-7.27(m, 1 H), 7.44-7.51 (m, 3 H), 8.40 (d, 2 H);HPLC (purity): 98.6%; Mass calculated forC₂₄H₂₅N₅OS −431.6, observed 432.1; R_{f} 0.67(Ethyl acetate: Hexane (3:7). |

### Example 56

### Synthesis of 4-(5-amino-3-tert-butyl-1H-pyrazol-1-yl)-2-(4-methoxyphenyl)-6-(methylthio)pyrimidine-5-carbonitrile

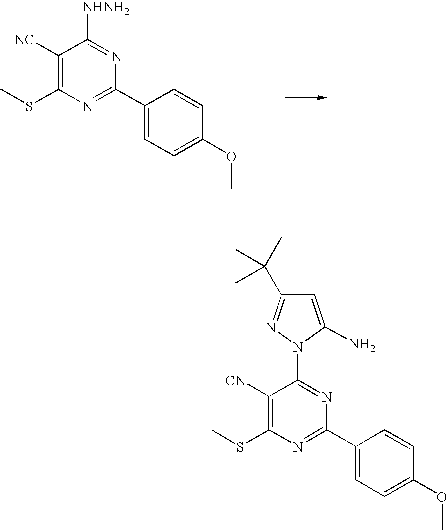

To a mixture of the hydrazine derivative shown above (60 mg, 2. 1 mmoles) and 4,4-dimethyl-3-oxopentanenitrile (52 mg, 4.2 mmoles) was added methanol (3 ml). The resulting solution was stirred for 4 hours at 80° C. Subsequently the reaction mixture was poured into water (50 ml) and dichloromethane (100 ml) was added. The organic layer was separated, dried over anhydrous sodium sulphate and concentrated at reduced pressure. The residue obtained was subjected to column chromatography using a gradient of methanol in dichloromethane (0-10%). Yield 22%; ¹H-NMR (CDCl₃) δ: 1.23-1.27 (s, 9H), 2.77 (s, 3H), 3.84 (s, 3H), 5.76 (s, 1H), 7.07 (d, 2H), 8.41 (d, 2H); HPLC (purity); HPLC (purity): 94.3%; Mass calculated for C₂₀H₂₂N₆OS 394.5, observed 395.1; R_{f}0.61 in chlorofrom:methanol (9:1).

### Details of the Assay Methods

Examples of pharmacological assays used for finding out the efficacy of the compounds of the present invention have been described below wherein their protocols and results are provided.

### In Vitro Measurement of Tumor Necrosis Factor Alpha (TNF-α)

This assay determines the effect of test compounds on the production of TNF-α in human Peripheral Blood Mononuclear Cells (PBMC). Compounds were tested for their ability to inhibit the activity of TNF-α in human PBMC. PBMC were isolated from blood (of healthy volunteers) using BD Vacutainer CPT™ (Cell preparation tube, BD Bio Science) and suspended in RPMI medium (Physiol. Res. 52: 593-598, 2003). The test compounds were pre-incubated with PBMC (0.5 million/incubation well) for 15 min. at 37° C. and then stimulated with Lipopolysaccharide (Escherichia coli: B4; 1 μg/ml) for 18 h at 37° C. in 5% CO₂. The levels of TNF-α in the cell culture medium were estimated using enzyme-linked immunosorbent assay performed in a 96 well format as per the procedure of the manufacturer (Cayman Chemical, Ann Arbor, USA). Representative result of TNF-α inhibition are shown in the Table I.

TABLE I

TNF-α Inhibition (%) at

| Example No. | Conc. (1 μM) | Conc. (10 μM) |
|---|---|---|
| 1 | 57.32 | 92.71 |
| 2 | 50.89 | 95.37 |
| 6 | NA | 88.00 |
| 10 | 20 | 9.7 |
| 13 | 13 | 71 |
| 39 | 19.19 | 34.02 |

### In Vitro Measurement of Interleukin-6 (IL-6)

This assay determines the effect of test compounds on the production of IL-6 in human PBMC (Physiol. Res. 52: 593-598, 2003). Compounds were tested for their ability to inhibit the activity of IL-6 in human PBMC. PBMC were isolated from blood using BD Vacutainer CPT™ Cell preparation tube (BD Bio Science) and suspended in RPMI medium. The test compounds were pre-incubated with PBMC (0.5 million/incubation well) for 15 min at 37° C. and then stimulated with Lipopolysaccharide (Escherichia coli: B4; 1 μg/ml) for 18 h at 37° C. in 5% CO₂. The levels of IL-6 in cell culture medium were estimated using enzyme-linked immunosorbent assay performed in a 96 well format as per the procedure of the manufacturer (Cayman Chemical, Ann Arbor, USA).

### Carrageenan Induced Paw Edema Test in Rat

The carrageenan paw edema test was performed as described by Winter et al (Proc. Soc. Exp. Biol. Med, 1962, 111, 544). Male wistar rats were selected with body weights equivalent within each group. The rats were fasted for 18 h with free access to water. The rats were dosed orally with the test compound suspended in the vehicle containing 0.25% carboxymethylcellulose and 0.5% Tween 80. The control rats were administered with vehicle alone. After an hour, the rats were injected with 0.1 ml of 1% Carrageenan solution in 0.9% saline into the sub-plantar surface of the right hind paw. Paw volume was measured using digital plethysmograph before and after 3 h of carrageenan injection. The average of foot swelling in drug treated animals was compared with that of the control animals. Anti-inflammatory activity was expressed as the percentage inhibition of edema compared with control group [Arzneim-Forsch/Drug Res., 43 (I), 1,44-50,1993; Otterness and Bliven, Laboratory Models for Testing NSAIDs, In Non-Steroidal Anti-Inflammatory Drugs.

### In Vitro Evaluation of Cyclooxygenase-2 (Cox-2) Inhibition Activity

The compounds of this invention exhibited in vitro inhibition of COX-2. The COX-2 inhibition activities of the compounds illustrated in the examples were determined by the following method.

### Human Whole Blood Assay

Human whole blood provides a protein and cell rich milieu appropriate for the study of the biochemical efficacy of anti-inflammatory compounds such as selective COX-2 inhibitors. Studies have shown that normal human blood does not contain the COX-2 enzyme. This correlates with the observation that COX-2 inhibitors have no effect on prostaglandin E₂ (PGE₂) production in normal blood. These inhibitors were active only after incubation of human blood with lipopolysaccharide (LPS), which induces COX-2 production in the blood.

Fresh blood was collected in tubes containing sodium heparin by vein puncture from healthy male volunteers. The subjects should have no apparent inflammatory conditions and should have not taken NSAIDs for at least 7 days prior to blood collection. Blood was preincubated with aspirin in vitro (12 μg/ml, at time zero) to inactivate COX-1 for 6 h. Then test compounds (at various concentrations) or vehicle were added to blood, the blood was stimulated with LPS B:4 (10 μg/ml) and incubated for another 18 h at 37° C. water bath. After which the blood was centrifuged, plasma was separated and stored at −80° C. (J. Pharmacol. Exp. Ther, 1994, 271, 1705; Proc. Natl. Acad. Sci. USA, 1999, 96, 7563). The plasma was assayed for PGE₂ using Cayman ELISA kit as per the procedure outlined by the manufacturer (Cayman Chemicals, Ann Arbor, USA).

### COX-1 and COX-2 Enzyme Based Assay

COX-1 and COX-2 enzyme based assays were carried out to check the inhibitory potential of the test compounds on the production of prostaglandin by purified recombinant COX-1/COX-2 enzyme (Proc. Nat. Acad. Sci. USA, 1991, 88, 2692-2696; J. Clin. Immunoassay, 1992, 15, 116-120). In this assay, the potential of the test compounds to inhibit the production of prostaglandin either by COX-1 or COX-2 from arachidonic acid (substrate) was measured. This was an enzyme based in vitro assay to evaluate selective COX inhibition with good reproducibility.

Arachidonic acid was converted to PGH₂ (Intermediate product) by COX1/COX-2 in the presence or absence of the test compound. The reaction was carried out at 37° C. and after 2 min it was stopped by adding 1M HCl. Intermediate product PGH₂ was converted to a stable prostanoid product PGF_{2α} by SnCl₂ reduction. The amount of PGF2α produced in the reaction was inversely proportional to the COX inhibitory potential of the test compound. The prostanoid product was quantified via enzyme immunoassay (EIA) using a broadly specific antibody that binds to all the major forms of prostaglandin, using Cayman ELISA kit as per the procedure outlined by the manufacturer (Cayman Chemicals, Ann Arbor, USA).

### Ulcerogenic Potential

In order to evaluate the compound's role on the ulcer formation, the animals were sacrificed and the stomach was taken out and flushed with 1% formalin. Animals (male wistar 200 g) were fasted for 18 hours with free access to water and the test compounds were suspended in 0.5% Tween 80 and 0.25% CMC (carboxymethylcellulose) solution to make a uniform suspension. After 4 hours of oral administration of test compounds, all the animals were sacrificed by cervical dislocation. The stomach was dissected carefully and filled up with a sterile saline solution and embedded in 6% formalin solution. Finally the stomach was cut longitudinally and ulcer lesions were observed with computerized stereomicroscope. The test compound treated groups were compared with the vehicle treated groups. Doses selected: 50, 100, 200 mg/kg (Marco Romano et al, Journal of clinical Investigation, 1992, 90, 2409-2421.)

### Inhibitory Action on Adjuvant Arthritis in Rats

Compounds were assayed for their activity on rat adjuvant induced arthritis model according to Theisen-Popp et al., (Agents Actions, 1994, 42, 50-55). 6 to 7 weeks old, wistar rats were weighed, marked and assigned to groups [a negative control group in which arthritis was not induced (non-adjuvant control), a vehicle-treated arthritis control group, test substance treated arthritis group]. Adjuvant induced arthritis was induced by an injection of 0.1 ml of Mycobacterium butyricum (Difco) suspended in mineral oil (5 mg/ml) into the sub-plantar region of the right hind paw (J. Pharmacol. Exp. Ther., 1998, 284, 714). Body weight, and paw volumes were measured at various days (0, 4, 14, 21) for all the groups. The test compound or vehicle was administered orally, beginning post injection of adjuvant (‘0’ day) and continued for 21 days (pre-treatment group). In the post-treatment group, the test compound or vehicle was administered starting from day 14^{th} to 21^{st} day. On day 21, body weight and paw volume of both right and left hind paws were taken. Spleen, and thymus weights were determined. In addition, the radiographs of both hind paws were taken to assess the tibio-tarsal joint integrity. Hind limb below the stifle joint was removed and fixed in 1% formalin saline for the histopathological assessment. At the end of the experiment, serum samples were analysed for inflammatory mediators. The presence or absence of lesions in the stomach was also observed.

Two-factor (‘treatment’ and ‘time’) analysis of variance with repeated measures on ‘time’ was applied to the percentage (%) changes for body weight and foot volumes. A post hoc Dunnett's test was conducted to compare the effect of treatments to vehicle control. A one-way analysis of variance was applied to the thymus and spleen weights followed by the Dunnett's test to compare the effect of treatments to vehicle. Dose-response curves for percentage inhibition in foot volumes on days 4, 14 and 21 were fitted by a 4-parameter logistic function using a nonlinear least Squares' regression. IC₅₀ was defined as the dose corresponding to a 50% reduction compared to the vehicle control and was derived by interpolation from the fitted 4-parameter equation.

### LPS Induced Sepsis for Measurement of TNF-α Inhibition in Mice

The LPS induced sepsis model in mice was performed as described by Les sekut et al (J Lab Clin Med, 1994, 124, 813-820). Female Swiss albino mice were selected and the body weights were equivalent within each group. The mice were fasted for 20 h with free access to water. The mice were dosed orally with the test compound suspended in vehicle containing 0.5% Tween 80 in 0.25% Carboxy-methylcellulose sodium salt. The control mice were administered the vehicle alone. After 30 minutes of oral dosing, mice were injected with 500 μg of Lipopolysaccharide (Escherichia coli, LPS: B4 from Siga) in phosphate buffer saline solution into the intraperitoneal cavity of the mice. After 90 min of LPS administration mice were bled via retro-orbital sinus puncture. Blood samples were stored overnight at 4° C. Serum samples were collected by centrifuging the samples at 4000 rpm for 15 minutes at 4° C. Immediately the serum samples were analysed for TNF-α levels using commercially available mouse TNF-α ELISA kit (Amersham Biosciences) and assay was performed by the manufacturer instruction.

### ED₅₀ Measurement of TNF Alpha Inhibition in Mice Sepsis Model

TABLE II

| Ex. | Mice Sepsis ED₅₀ mg/kg |
|---|---|
| 1 | 3.1 |

### Inhibitory Activity in IBD-DSS Model

DSS Induced colitis test was performed as described by Axelsson et al., 1998. Male BALB/c mice were selected in the age of 7-8 weeks for the study. Colitis in mice was induced by providing DSS (2%) in the drinking water from day 1 to 6. Mice were dosed from Day 1 to 6 with test compound suspended in vehicle containing 0.25% carboxymethylcellulose and 0.5% Tween 80. The control animals received vehicle alone. Body weight and disease activity index was recorded daily during the experiment. After 6 days of treatment, animals were sacrificed; colon weight and colon length was recorded. Representative results are shown in the table III.

TABLE III

| Ex. | IBD-DSS Model % Inhibition of DAI at 50 mg/kg |
|---|---|
| 1 | 47 |

### Inhibitory Activity in Psoriasis Model

Oxazolone induced dermatitis in mice was performed as described in the literature. Female BALB/c were selected in the age of 6-7 weeks for the study and 20-25g. Mice were sensitized with oxazolone (15%) from Day 1 to day 6 by applying it on the shaved abdomen. Elicitation was done with oxazolone (2%) on the ear on day 7. Test compounds were applied topically on the ear 15 min and 6 h post oxazolone application on day 7. 24 h after oxazolone application, ear thickness is measured and ear were excised under anesthesia and weighed.

PDE4 Activity

PDE4 inhibition was measured by following a literature assay procedure (Cortizo J et al., J. Pharmacol., 1993, 108, 562-568). The assay method involves the following conditions.

Source: Human U937 cells
Substrate: 1.01 μM [³H] camp+camp

### Vehicle: 1% DMSO

### Pre-Incubation Time/Temperature: 15 min at 25° C.

### Incubation Time/Temperature. 20 min at 25° C.

Incubation buffer: 50 mM Tris-HCl, pH 7.5, 5 mM MgCl₂
Quantitation method: Quantitation of [³H] Adenosine
Significance criteria: 50% of max stimulation or inhibition
The results are tabulated as shown in the table IV.

TABLE IV

| Ex. | PDE4 inhibition at 10 μM (%) |
|---|---|
| 1 | 61 |

### 5-LOX Activity

The assay method involves the following conditions.
Source: Human PBML cells
Substrate: Arachidonic acid

### Vehicle: 1% DMSO

### Pre-incubation Time/Temp.: 15 min at 37° C.

### Incubation Time/Temp.: 15 min at 37° C.

Incubation buffer: HBSS (Hank's balanced salt solution)
Quantitation method: EIA quantitation of LTB4
Significance criteria: >50% of maximum stimulation or inhibition
The results are tabulated as shown in the table V.

TABLE V

| Ex. | 5-LOX inhibition at 10 μM (%) |
|---|---|
| 1 | 94 |

### Thromboxane Synthase Activity

The assay method involves the following conditions.

### Source: Human Platelets

### Substrate: 10 μM PGH2

### Vehicle: 1% DMSO

### Pre-incubation Time/Temp.: 15 min at 25° C.

### Incubation Time/Temp.: 3 min at 25° C.

Incubation buffer: 10 mM Tris-HCl, pH 7.4
Quantitation method: EIA quantitation of TxB2
Significance criteria: >50% of maximum stimulation or inhibition
The results are tabulated as shown in the table VI.

TABLE VI

| Ex. | Thromboxane synthase inhibition at 10 μM (%) |
|---|---|
| 1 | 96 |

### Anti-Cancer Screen

Experimental drugs are screened for anti-cancer activity in three cell lines for their GI₅₀, TGI and LC₅₀ values (using 5 concentrations for each compound). The cell lines are maintained in DMEM containing 10% fetal bovine serum. 96 well microtiter plates are inoculated with cells in 100 μL for 24 hours at 37° C., 5% CO₂, 95% air and 100% relative humidity. 5000 HCT116 cells/well, 5000 NCIH460 cells/well, 10000 U251 cells/well and 5000 MDAMB231 cells/well are plated. A separate plate with these cell lines is also inoculated to determine cell viability before the addition of the compounds (T₀).

### Addition of Experimental Drugs

Following 24-h incubation, experimental drugs are added to the 96 well plates. Each plate contains one of the above cell lines and the following in triplicate: 5 different concentrations (0.01, 0.1, 1, 10 and 100 μM) of 4 different compounds, appropriate dilutions of a cytotoxic standard and control (untreated) wells. Compounds are dissolved in dimethylsulfoxide (DMSO) to make 20 mM stock solutions on the day of drug addition and frozen at −20° C. Serial dilutions of these 20 mM stock solutions are made in complete growth medium such that 100 μL of these drug solutions in medium, of final concentrations equaling 0.01, 0.1, 1, 10 and 100 μM can be added to the cells in triplicate. Standard drugs whose anti-cancer activity has been well documented and which are regularly used are doxorubicin and SAHA.

### End-Point Measurement

Cells are incubated with compounds for 48 h followed by the addition of 10 μL 3-(4,5-Dimethyl-2-thiazolyl)-2,5-diphenyl-2H-tetrazolium (MTT) solution per well and a subsequent incubation at 37° C., 5% CO₂, 95% air and 100% relative humidity, protected from light. After 4 h, well contents are aspirated carefully followed by addition of 150 μL DMSO per well. Plates are agitated to ensure solution of the formazan crystals in DMSO and absorbance read at 570 nm.

### Calculation of GI₅₀, TGI and LC₅₀

Percent growth is calculated for each compound's concentration relative to the control and zero measurement wells (T₀; viability right before compound addition). If a test well's O.D. value is greater than the T₀ measurement for that cell line

% Growth=(test−zero)/(control−zero)×100

If a test well's O.D. value is lower than the T₀ measurement for that cell line, then

% Growth=(test−zero)/zero×100

Plotting % growth versus experimental drug concentration, GI₅₀ is the concentration required to decrease % growth by 50%; TGI is the concentration required to decrease % growth by 100% and LC₅₀ is the concentration required to decrease % growth by 150%.

Screening of selected compounds in three anti-cancer cell lines resulted in a hit compound whose data is tabulated below.

| | NCIH460 | HCT116 | U251 | |
|---|---|---|---|---|
| Example | GI₅₀ | GI₅₀ | GI₅₀ | Mean GI₅₀ |
| 6 | 10.10 | 4.90 | 0.92 | 5.3 |

1. Compounds of formula (I), 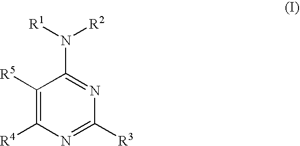
their derivatives, analogs, tautomeric forms, stereoisomers, polymorphs, hydrates, solvates, pharmaceutically acceptable salts, pharmaceutical compositions, metabolites and prodrugs thereof, wherein,
R¹ and R² independently represent hydrogen, amino group, optionally substituted groups selected from linear or branched alkyl, cycloalkyl, alkylsulfonyl, aryl, heteroaryl; nitrogen containing saturated or unsaturated heterocyclyl ring; or R¹ and R² can together with the nitrogen atom to which they are attached form an optionally substituted saturated or unsaturated cyclic ring;
R³ represents optionally substituted groups selected from linear or branched alkyl, alkylthio, aryl and heteroaryl;
R⁴ represents optionally substituted groups selected from linear or branched alkyl, alkylthio, alkylsulfonyl, alkylsulfinyl, aryl and heteroaryl;
R⁵ represents hydrogen, hydroxyl, halogen, nitro, amino, cyano, amide, carboxylic acid and its derivatives, optionally substituted groups selected from linear or branched alkyl.

2. A compound of the formula (Ia), 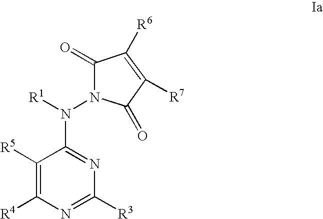
wherein:
R¹ represents hydrogen; optionally substituted groups selected from linear or branched alkyl group comprising methyl, ethyl, n-propyl, isopropyl, n-butyl, isobutyl, t-butyl, n-pentyl, isopentyl and hexyl; cycloalkyl group comprising cyclopropyl and cyclobutyl; amino; alkylsulfonyl group comprising methylsulfonyl and ethylsulfonyl;
R³ represents halogen comprising fluorine, chlorine, bromine and iodine; substituted or unsubstituted alkyl group; haloalkyl group comprising chloromethane, chloroethane, trifluoromethane, trifluoroethane, dichloromethane and dichloroethane; optionally substituted groups selected from linear or branched alkyl; alkoxy group comprising methoxy and ethoxy; alkylthio group comprising methylthio and ethylthio; alkylsulfinyl group comprising methylsulfinyl, ethylsulfinyl; aryl group comprising phenyl and naphthyl; heterocyclyl group comprising pyrrolidinyl, thiazolidinyl, oxazolidinyl, morpholinyl, thiomorpholinyl, piperidinyl and piperazinyl; and heteroaryl group comprising pyridyl, thienyl, furyl, pyrrolyl, oxazolyl, thiazolyl, imidazolyl, pyrazolyl, oxadiazolyl, thiadiazolyl, tetrazolyl, pyrimidinyl benzopyranyl, benzimidazolyl, benzoxazolyl, benzothiazolyl, benzopyrrolyl, benzoxadiazolyl, benzothiadiazolyl, quinolinyl, isoquinolinyl, benzothienyl, benzofuranyl and indolyl;
R⁴ represents optionally substituted groups selected from linear or branched alkyl; alkylthio; alkylsulfonyl; alkylsulfinyl group comprising methylsulfinyl and ethylsulfinyl; aryl and heteroaryl;
R⁵ represents hydrogen; hydroxyl; halogen; nitro; cyano; amide; heterocyclyl groups comprising substituted or unsubstituted tetrazolyl carboxylic acid and its derivatives; optionally substituted groups selected from linear or branched alkyl and amino;
R⁶ and R⁷ represents hydrogen; halogen; nitro; haloalkyl; optionally substituted groups selected from linear or branched alkyl; amino; aryl; heteroaryl or R⁶ and R⁷ can together form a optionally substituted saturated or unsaturated cyclic ring comprising cycloalkyl; aryl and heteroaryl;
when the groups R¹, R², R³, R⁴, R⁵, R⁶ and R⁷ have one or more substitutents, the substituents are selected from halogen; haloalkyl; oxo; nitro; hydroxyl; carboxylic acid; ester; amide; alkyl; alkoxy; amino; aminosulfonyl; heterocyclylalkyl; heterocyclylsulfonyl; alkylthio; mercapto; aryl; heteroaryl and heteroarylalkyl groups; which in turn are optionally substituted by halogen; alkyl; alkoxy; aryl and heteroaryl.

3. The compound according to claim 1 selected from the group consisting of:
4-(3-Methyl-2,5-dioxo-2,5-dihydro-1H-pyrrol-1-ylamino)-6-(methylthio)-2-phenylpyrimidine-5-carbonitrile;
4-(Thiomethyl)-2-(4-methoxyphenyl)-6-(3-methyl-2,5-dioxo-2,5-dihydro-1H-pyrrol-1-ylamino)pyrimidine-5-carbonitrile;
4-Hydrazinyl-2-(4-methoxyphenyl)-6-(methylthio)pyrimidine-5-carbonitrile;
5-(5-Cyano-4-hydrazinyl-6-(methylthio)pyrimidin-2-yl)-2-methoxybenzenesulfonamide;
4-(3-Methyl-2,5-dioxo-2,5-dihydro-1H-pyrrol-1-ylamino)-6-(methylthio)-2-(2,4,6-trimethoxyphenyl)pyrimidine-5-carbonitrile;
5-(5-Cyano-4-(3-methyl-2,5-dioxo-2,5-dihydro-1H-pyrrol-1-ylamino)-6-(methylthio)pyrimidin-2-yl)-2-methoxybenzenesulfonamide;
4-Hydrazinyl-6-(methylthio)-2-(4-(trifluoromethyl)phenyl)pyrimidine-5-carbonitrile; 3-(5-Cyano-4-hydrazinyl-6-(methylthio)pyrimidin-2-yl)benzenesulfonamide;
4-(3-Methyl-2,5-dioxo-2,5-dihydro-1H-pyrrol-1-ylamino)-6-(methylthio)-2-(4-trifluoromethyl)phenyl)pyrimidine-5-carbonitrile;
3-(5-Cyano-4-(3-methyl-2,5-dioxo-2,5-dihydro-1H-pyrrol-1-ylamino)-6-(methylthio)pyrimidin-2-yl)benzenesulfonamide;
2-(4-Chlorophenyl)-4-(3-methyl-2,5-dioxo-2,5-dihydro-1H-pyrrol-1-ylamino)-6-(methylthio)pyrimidine-5-carbonitrile;
2-(4-(Dimethylamino)phenyl)-4-(3-methyl-2,5-dioxo-2,5-dihydro-1H-pyrrol-1-ylamino)-6-(methylthio)pyrimidine-5-carbonitrile;
4-Hydrazinyl-6-(methylthio)-2-(thiophen-2-yl)pyrimidine-5-carbonitrile;
4-(3-Methyl-2,5-dioxo-2,5-dihydro-1H-pyrrol-1-ylamino)-6-(methylamino)-2-(thiophen-2-yl)pyrimdine-5-carbonitrile;
4-(Thiomethyl)-2-(3-ethoxycarbonyl-phenyl)-6-(3-methyl-2,5-dioxo-2,5-dihydro-1H-pyrrol-1-ylamino)pyrimidine-5-carbonitrile;
Ethyl-3-(5-cyano-4-(5-methyl-3,6-dioxo-2,3-dihydropyridazin-1(6H)-yl)-6-(methylthio)pyrimidin-2-yl)benzoate;
4-(3-Methyl-2,5-dioxo-2,5-dihydro-1H-pyrrol-1 ylamino)-6-(methylthio)-2-p-tolylpyrimidine-5-carbonitrile;
2-(2-Chloropyridin-3-yl)-4-(3-methyl-2,5-dioxo-2,5-dihydro-1H-pyrrol-1-ylamino)-6-(methylthio)pyrimidine-5-carbonitrile;
4-(3-Methyl-2,5-dioxo-2,5-dihydro-1H-pyrrol-1-ylamino)-6-(methylthio)-2-(4-(methylthio)phenyl)pyrimidine-5-carbonitrile;
2-(4-((2-Methyl-1H-imidazol-1-yl)methyl)phenyl)-4-(3-methyl-2,5-dioxo-2,5-dihydro-1H-pyrrol-1-ylamino)-6-(methylthio)pyrimidine-5-carbonitrile;
2-(2-Chlorophenyl)-4-(3-methyl-2,5-dioxo-2,5-dihydro-1H-pyrrol-1-ylamino)-6-(methylthio)pyrimidine-5-carbonitrile;
2-(3,4-Dihydroxyphenyl)-4-(3-methyl-2,5-dioxo-2,5-dihydro-1H-pyrrol-1-ylamino)-6-(methylthio)pyrimidine-5-carbonitrile;
2-(2,4-Dichlorophenyl)-4-(3-methyl-2,5-dioxo-2,5-dihydro-1H-pyrrol-1-ylamino)-6-(methylthio)pyrimidine-5-carbonitrile;
2-(2-Fluorophenyl)-4-(3-methyl-2,5-dioxo-2,5-dihydro-1H-pyrrol-1-ylamino)-6-(methylthio)pyrimidine-5-carbonitrile;
2-(3,4-Difluorophenyl)-4-(3-methyl-2,5-dioxo-2,5-dihydro-1H-pyrrol-1-ylamino)-6-(methylthio)pyrimidine-5-carbonitrile;
2-(4-Hydroxyphenyl)-4-(3-methyl-2,5-dioxo-2,5-dihydro-1H-pyrrol-1-ylamino)-6-(methylthio)pyrimidine-5-carbonitrile;
4-(3,4-Dichloro-2,5-dioxo-2,5-dihydro-1H-pyrrol-1-ylamino)-2-(4-methoxyphenyl)-6-(methylthio)pyrimidine-5-carbonitrile;
4-(3,4-Dimethyl-2,5-dioxo-2,5-dihydro-1H-pyrrol-1-ylamino)-2-(4-methoxyphenyl)-6-(methylthio)pyrimidine-5-carbonitrile;
2-(2-Methoxyphenyl)-4-(3-methyl-2,5-dioxo-2,5-dihydro-1H-pyrrol-1-ylamino)-6-(methylthio)pyrimidine-5-carbonitrile;
4-(3-Methyl-2,5-dioxo-2,5-dihydro-1H-pyrrol-1-ylamino)-6-(methylthio)-2-(naphthalen-1-yl)pyrimidine-5-carbonitrile;
2-Methyl-4-(3-methyl-2,5-dioxo-2,5-dihydro-1H-pyrrol-1-ylamino)-6-(methylthio)-2-(naphthalen-1-yl)pyrimidine-5-carbonitrile;
4-(3,4-Dimethyl-2,5-dioxo-2,5-dihydro-1H-pyrrol-1-ylamino)-6-(methylthio)-2-(phenyl)pyrimidine-5-carbonitrile;
4-(3,4-Dichloro-2,5-dioxo-2,5-dihydro-1H-pyrrol-1-ylamino)-6-(methylthio)-2-(phenyl)pyrimidine-5-carbonitrile;
4-(1,3-Dioxoisoindolin-2-ylamino)-6-(methylthio)-2-ylamino)-6-(methylthio)-2-phenylpyrimidine-5-carbonitrile;
4-(3-Methyl-2,5-dioxo-2,5-dihydro-1H-pyrrol-1-ylamino)-6-(methylthio)-2-m-tolylpyrimidine-5-carbonitrile;
4-(Thiomethyl)-2-(thiomethyl)-6-(3-methyl-2,5-dioxo-2,5-dihydro-1H-pyrrol-1-ylamino)pyrimidine-5-carbonitrile;
1-(6-(3-(Aminothioperoxy)-4-methylphenyl)-2-methylpyrimidin-4-ylamino)-3-methyl-1H-pyrrole-2,5-dione;
3-Methyl-1-(2-methyl-6-phenylpyrimidin-4-ylamino)-1H-pyrrole-2,5-dione;
2-(4-Methoxyphenyl)-4-(methylthio)-6-(4-(pyridin-2-yl)piperazin-1-yl)pyrimidine-5-carbonitrile;
4-(1H-imidazol-1-yl)2-(4-methoxyphenyl)-6-(methylthio)pyrimidine-5-carbonitrile;
2-(4-Methoxyphenyl)-4-(methylthio)-6-(piperazin-1-yl)pyrimidine-5-carbonitrile;
4-(Methylthio)-6-morpholino-2-phenylpyrimidine-5-carbonitrile;
2-(4-Methoxyphenyl)-4-(methylthio)-6-morpholinopyrimidine-5-carbonitrile;
4-(1H-imidazol-1-yl)-6-(methylthio)-2-phenylpyrimidine-5-carbonitrile;
2-(4-Methoxyphenyl)-4-(methylthio)-6-(4-nitro-1H-imidazol-1-yl)pyrimidine-5-carbonitrile;
5-(5-Cyano-4-(methylthio)-6-morpholinopyrimidin-2-yl)-2-methoxybenzenesulfonamide;
2-(4-Methoxy-3-(morpholinsulfonyl)phenyl)-4-(methylthio)-6-morpholinopyrimidine-5-carbonitrile;
5-(5-Cyano-4-(methylthio)-6-(4-(pyridin-2-yl)piperazin-1-yl)pyrimidin-2-yl)-2-methoxybenzenesulfonamide;
5-(5-Cyano-4-(cyclopropylamino)-6-(methylthio)pyrimidin-2-yl)-benzenesulfonamide;
5-(5-Cyano-4-(cyclopropylamino)-6-(methylthio)pyrimidin-2-yl)-2-methoxybenzenesulfonamide;
4-(Methylthio)-2-phenyl-6-(piperazin-1-yl)pyrimidine-5-carbonitrile;
4-(Methylthio)-2-phenyl-6-(4-(pyridine-2-yl)piperazin-1-yl)pyrimidine-5-carbonitrile;
4-(Methylthio)-2-phenyl-6-(4-(pyridin-2-ylmethyl)piperazin-1-yl)pyrimidine-5-carbonitrile;
Ethyl 3-(2-(2-chlorophenyl)-5-cyano-6-(methylthio)pyrimidin-4-ylamino)-4 hydroxy-5-methoxybenzoate;
4-(4-Methoxybenzyl)piperazin-1-yl)-6-(methylthio)-2-phenylpyrimidine-5-carbonitrile; and
4-(5-Amino-3-t-butyl-1H-pyrazol-1-yl)-2-(4-methoxyphenyl)-6-(methylthio)pyrimidine-5-carbonitrile;

4. A process for the preparation of heterocyclic compounds of formula (I) as claimed in claim 1, which comprises:
reacting optionally substituted 4-chloro pyrimidines of formula G₁, 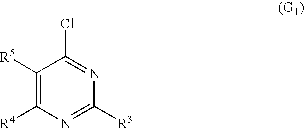
with appropriate heterocyclyl compound or hydrazine and its derivatives.

5. A process for the preparation of heterocyclic compounds of formula (Ia) as claimed in claim 2, which comprises:
reacting optionally substituted 4-chloro pyrimidines of formula G₁ with appropriate hydrazine and its derivatives followed by reaction with cyclic anhydrides.

6. A process for the preparation of heterocyclic compounds of formula (I) as claimed in claim 4, wherein R² is 3-methyl-1H-pyrrole-2,5-dione; R³ is methyl or optionally substituted phenyl; R⁴ is thiomethyl or optionally substituted phenyl and R⁵ is hydrogen or carbonitrile.

7. A pharmaceutical composition comprising a compound of formula (I) as claimed in claim 1, as an active ingredient along with a pharmaceutically acceptable carrier, diluent, excipient or solvate.

8. The pharmaceutical composition comprising a compound of formula (Ia) as claimed in claim 2, as an active ingredient along with a pharmaceutically acceptable carrier, diluent, excipient or solvate.

9. The pharmaceutical composition as claimed in claim 7 wherein the said composition is in the form of a tablet, capsule, powder, syrup, solution, aerosol or suspension.

10. The pharmaceutical composition as claimed in claim 7 wherein the amount of the compound of formula (I) in the composition is less than 70% by weight.

11. A method of treatment of a pain disorder, inflammation, and immunological diseases in a mammal comprising administering an effective amount of, a compound according to claim 1 to the mammal in need thereof.

12. A method of treatment of rheumatoid arthritis; osteoporosis; multiple myeloma; uveititis; acute and chronic myelogenous leukemia; ischemic heart disease; atherosclerosis; cancer; ischemic-induced cell damage; pancreatic beta cell destruction; osteoarthritis; rheumatoid spondylitis; gouty arthritis; inflammatory bowel disease; adult respiratory distress syndrome (ARDS); psoriasis; Crohn's disease; allergic rhinitis; ulcerative colitis; anaphylaxis; contact dermatitis; muscle degeneration; cachexia; asthma; bone resorption diseases; ischemia reperfusion injury; brain trauma; multiple sclerosis; sepsis; septic shock; toxic shock syndrome; fever and myalgias due to infection in a mammal comprising administering an effective amount of a compound of formula (I) according to claim 1 to the mammal in need thereof.

13. A method of lowering plasma concentrations of anyone or a combination or all of TNF-α, IL-1β, and IL-6 comprising administering an effective amount of a compound according to claim 1 to the mammal in need thereof.

14. A method for inhibiting production of cytokines as selected from TNF-α, IL-1β, L-6 and IL-12 by method comprising administering the compound of the formula (I) as claimed in claim 1.

15. A method of treating immunological diseases, those mediated by cytokines comprising TNF-α, IL-1β, IL-6 and IL-12 comprising administering an effective amount of a compound according to claim 1 to the mammal in need thereof.

16. A method of treating inflammatory diseases mediated by thromboxane synthase, comprising administering an effective amount of a compound according to claim 1 to the mammal in need thereof.

17. A method of treating inflammatory diseases mediated by Lipoxygenases, particularly 5-lipoxygenase, comprising administering an effective amount of a compound according to claim 1 to the mammal in need thereof.

18. A method of treating inflammatory diseases mediated by PDE4 inhibitors comprising administering an effective amount of a compound according to claim 1 to the mammal in need thereof.

19. A method of lowering plasma concentrations of anyone or a combination or all of TNF-α, IL-1β, and IL-6 comprising administering an effective amount of a compound according to claim 3 to the mammal in need thereof.

20. A method for inhibiting production of cytokines as selected from TNF-α, IL-1β, IL-6 and IL-12 by method comprising administering the compound of the formula (I) as claimed in claim 3.

21. A method of treating immunological diseases, those mediated by cytokines comprising TNF-α, IL-1β, IL-6 and IL-12 comprising administering an effective amount of a compound according to claim 3 to the mammal in need thereof.

22. A method of treating inflammatory diseases mediated by thromboxane synthase, comprising administering an effective amount of a compound according to claim 3 to the mammal in need thereof.

23. A method of treating inflammatory diseases mediated by Lipoxygenases, particularly 5-lipoxygenase, comprising administering an effective amount of a compound according to claim 3 to the mammal in need thereof.

24. A method of treating inflammatory diseases mediated by PDE4 inhibitors comprising administering an effective amount of a compound according to claim 3 to the mammal in need thereof.
